(19) United States
(12) Patent Application Publication
(10) Pub. No.: US 2023/0121636 A1
(43) Pub. Date: Apr. 20, 2023

(54) UPPER LAYER SYSTEM FOR ABSORBENT ARTICLE

(71) Applicant: The Procter & Gamble Company, Cincinnati, OH (US)

(72) Inventors: Pietro CECCHETTO, Fairfield, OH (US); Misael Omar AVILES, Hamilton, OH (US); Gerard A. VIENS, Wyoming, OH (US); Olivia Maeve Newman, Cincinnati, OH (US); Jose Enrique BETANCOURT, West Chester, OH (US)

(21) Appl. No.: 17/964,997

(22) Filed: Oct. 13, 2022

Related U.S. Application Data
(60) Provisional application No. 63/256,164, filed on Oct. 15, 2021.

(51) Int. Cl. A61F 13/513; A61F 13/512

(52) U.S. Cl. CPC A61F 13/513; A61F 13/5125; A61F 2013/51383; A61F 2013/51011

(57) ABSTRACT

A feminine hygiene pad is disclosed. The pad may include a topsheet comprising a nonwoven web, and a fluid management layer beneath the topsheet. The web may include bicomponent staple fibers having an average denier of 1.5 to 2.5 and a sheath-core configuration, have a basis weight of about 18 gsm to 40 gsm, and a plurality of randomly-distributed inter-fiber bonds wherein sheaths of adjacent fibers are fusion-bonded together without compression. The sheaths may comprise PE and the cores may comprise PET, in a weight ratio of 40:60 to 60:40. The web may be formed predominantly of hydrophobic fibers, or the web may include a blend of hydrophilic fibers and hydrophobic fibers, in a weight ratio of 30:70 to 70:30, wherein fiber hydrophilicity is effected by application of a surface treatment composition. The topsheet may bear an application of an anti-stick agent and/or have a pattern of apertures therethrough.

### CROSS REFERENCE TO RELATED APPLICATION

This application claims the benefit of U.S. Provisional Application No. 63/256,164, filed Oct. 15, 2021, the substance of which is incorporated herein by reference.

### BACKGROUND

Absorbent articles of various designs have been used for many years for the purpose of intercepting, capturing, containing and absorbing bodily exudates including menstrual fluid, urine and feces, for the purpose of managing the exudate to avoid soiling of underwear, outer clothing, bedding, etc.

One type of absorbent article, the feminine hygiene pad (also known as a “sanitary napkin,” “menstrual pad,” etc.) has been used by women to manage discharge of menstrual fluid during menses. A typical feminine hygiene pad includes a liquid permeable topsheet which forms the body/wearing-facing surface of the pad; a liquid impermeable backsheet which forms the outward-facing surface of the pad and serves as a barrier to prevent fluid absorbed by the pad from migrating to and exiting the outward-facing surface of the pad; and an absorbent structure which serves fluid handling functions in any desired combination of lesser or greater extents, including capture, distribution, absorption, containment and storage, during the desired duration of wear/use of the pad. The typical pad is configured, shaped and sized to be placed inside the intended user's underpants in the crotch region thereof. Many pads include one or more deposits of adhesive on the outward-facing surface of the backsheet, to enable the wearer to affix the pad to the inside surface of the underwear to help hold it in a suitable position during wear/use.

A variety of materials have been developed and used to form topsheets for feminine hygiene pads.

In some examples, a topsheet may be formed of a polymeric film having a pattern of apertures formed therein. The apertures serve to allow discharged fluid to pass through the topsheet to absorbent components of the pad disposed therebeneath. Some segments of the consumer market, however, may not prefer film topsheets for various reasons which may include their feel against the skin.

In other examples, a topsheet may be formed of a fibrous nonwoven web material. Myriad combinations of features of a nonwoven are possible. The fibrous constituents may be relatively long fibers of indeterminate and varying lengths, or may be staple fibers. They may include natural fibers (e.g., cotton fibers); semi-synthetic fibers (e.g., regenerated cellulose (e.g., rayon, viscose, lyocell, etc.)) fibers. They may include single component or multicomponent fibers. The fibers may be relatively straight, or curled or crimped. Spun fibers may be spun from a variety of thermoplastic polymer components. A precursor batt or accumulation of constituent fibers may be consolidated, and the fibers held together, whereby the web has fabric-like characteristics and structural integrity, imparted by a variety of mechanisms including fiber entanglement, fiber-to-fiber fusion bonding, fiber-to-fiber binder/adhesive bonding, etc. Differing types of fibers of any desired selection and blended in any desired weight ratio to constitute the nonwoven web. Any portion or all of the constituent fibers or the nonwoven web may be treated to impart or increase hydrophobicity or hydrophilicity, or any combination or arrangement thereof, to the fibers' surfaces.

Generally, it is desired by users that the topsheet of a feminine hygiene pad:
readily admit menstrual fluid and facilitate its rapid passage in a z-direction downward therethrough (rapid acquisition), to underlying absorbent components;
resist x-y direction spreading of, and staining by, menstrual fluid;
resist reacquiring fluid from underlying absorbent components, and allowing it to move in a z-direction upwardly therethrough (rewetting) back to the wearer-facing surface (causing a wet feel for the wearer and/or a perception of incomplete absorption and less effective protection);
conceal, to the greatest extent practical, menstrual fluid that has been absorbed by the underlying absorbent components, from view therethrough (for perceptions of effective absorption and cleanliness); and
feel soft and comfortable against the wearer's skin, and dry, including after a fluid insult.

These objectives are in conflict to some extent. For example, a topsheet of nonwoven web material that readily wicks fluid downward in a z-direction may also tend to retain the fluid to some extent, or to readily wick it back upward in a z-direction (rewetting), or to wick it along x-y directions, causing spreading. Conversely, a topsheet that resists z-direction fluid movement from the absorbent components to the wearer-facing surface may also resist x-y direction fluid movement/wicking, but may also not readily admit fluid and pass it down to the absorbent components (slow acquisition). A web that has substantial permeability (provided by, e.g., large pore volumes) may readily admit and pass fluid therethrough, but may be insufficiently mechanically robust for processing on a converting line, or may be insufficiently opaque or insufficiently substantial in appearance to be acceptable to consumers/users. A variety of approaches have been attempted, through myriad combinations of the different components, configurations and features identified above, to meet conflicting objectives as best as possible. However, opportunity for improvement remains.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1A is a schematic plan view (along a z-direction) depiction of an example of an absorbent article.

FIG. 1B is a schematic plan view (along a z-direction) depiction of examples of absorbent core component layers of the absorbent article depicted in FIG. 1A.

FIG. 2 is a schematic depiction of examples of an arrangement of equipment and a process that may be configured to manufacture a fluid management layer.

FIG. 3 is a schematic depiction of an example of a cross section, taken along a z-direction plane, of a fluid management layer.

FIG. 4 is a plan view (along a z-direction) image of a portion of a nonwoven web material having a pattern of apertures therethrough.

FIG. 5 is a plan view (along a z-direction) magnified image of a portion of a nonwoven web material having an aperture therethrough.

FIG. 6 is a top view of a strikethrough plate used in the Acquisition Time and Rewet Measurement Method described herein.

FIG. 7 is a bottom view of the strikethrough plate used in the Acquisition Time and Rewet Measurement Method described herein.

FIG. 8A is a cross section view of the strikethrough plate used in the Acquisition Time and Rewet Measurement Method described herein, taken along a plane defined by the z-direction and line A-A shown in FIG. 6.

FIG. 8B is a cross section view of the strikethrough plate used in the Acquisition Time and Rewet Measurement Method described herein, taken along a plane defined by the z-direction and line B-B shown in FIG. 6.

FIGS. 9A and 9B are charts presenting second acquisition times measured for samples of 10 prototype feminine hygiene pads, measured using the Acquisition Time and Rewet Measurement Method described herein.

FIGS. 10A and 10B are charts presenting sums of surface free fluid (SFF) and rewet measured for samples of 10 prototype feminine hygiene pads, measured using the Acquisition Time and Rewet Measurement Method described herein.

### DESCRIPTION OF EXAMPLES

Definitions

As used herein, the following terms shall have the meaning specified thereafter:

“Absorbent article” refers to wearable devices, which absorb and/or contain liquid, and more specifically, refers to devices, which are placed against or in proximity to the body of the wearer to absorb and contain the various exudates discharged from the body. Absorbent articles can include diapers, training pants, adult incontinence undergarments (e.g., liners, pads and briefs) and/or feminine hygiene products, including feminine hygiene pads (also known as, for example, “sanitary napkins”, “menstrual pads”, “panty liners”, etc.).

The term “integrated” as used herein is used to describe fibers of a nonwoven material which have been intertwined, entangled, and/or pushed/pulled in a positive and/or negative Z-direction (direction of the thickness of the nonwoven material). Some exemplary processes for integrating fibers of a nonwoven web include spunlacing and needlepunching. Spunlacing (also known as “hydroentangling” or (“hydroenhancing”) uses a plurality of high pressure water jets directed at a precursor batt or accumulation of fibers being conveyed along a machine direction, to entangle the fibers. Needlepunching (also known as “needling”) involves the use of specially-featured needles to mechanically push and/or pull fibers, of a precursor batt or accumulation of fibers, in a z-direction, to entangle them with other fibers in the batt or accumulation.

The term “carded” as used herein is used to describe structural features of the fluid management layers described herein. A carded nonwoven web is formed of fibers which are cut to a specific finite length, otherwise known as “staple length fibers.” Staple length fibers may be of any selected length. For example, staple length fibers may be cut to a length of up to 120 mm, to a length as short as 10 mm. However, if fibers of a particular group are staple length fibers, then the length of each of the fibers in the carded nonwoven is approximately the same, i.e. the staple length. Where fibers of more than one composition are included in a nonwoven web, for example, a web including polypropylene fibers and viscose fibers, the length of each fiber of the same composition may be substantially the same, while the respective staple fiber lengths of the respective fiber compositions may differ.

In contrast to staple fibers, filaments such as those produced by spinning, e.g., in a spunbond or meltblown nonwoven web manufacturing processes, are not ordinarily staple length fibers. Instead, these filaments are sometimes characterized as “continuous” fibers, meaning that they are of a relatively long and indeterminate length, not cut to a specific length following spinning, as their staple fiber counterparts are.

“Lateral”—with respect to an absorbent article such as a feminine hygiene pad, or a component thereof, refers to a direction parallel to a horizontal line tangent to the front surfaces of the upper portions of wearer's legs proximate the torso, when the pad is being worn normally and the wearer has assumed an even, square, normal standing position. A “width” dimension of any component or feature of an article such as a feminine hygiene pad is measured along the lateral direction. When the article or component thereof is laid out flat on a horizontal surface, the “lateral” direction corresponds with the lateral direction relative the structure when it is worn, as defined above. With respect to an article such as a feminine hygiene pad that is opened and laid out flat on a horizontal planar surface, “lateral” refers to a direction perpendicular to the longitudinal direction and parallel to the horizontal planar surface.

The “lateral axis” of an absorbent article such as a feminine hygiene pad or component thereof is a lateral line lying in an x-y plane and equally dividing the length of the pad or the component when it is laid out flat on a horizontal surface. A lateral axis is perpendicular to a longitudinal axis.

“Longitudinal”—with respect to an absorbent article such as a feminine hygiene pad, or a component thereof, refers to a direction perpendicular to the lateral direction. A “length” dimension of any component or feature of the article is measured along the longitudinal direction from its forward extent to its rearward extent. When an article such as a feminine hygiene pad or component thereof is laid out flat on a horizontal surface, the “longitudinal” direction is perpendicular to the lateral direction relative the pad when it is worn, as defined above.

The “longitudinal axis” of a feminine hygiene pad or component thereof is a longitudinal line lying in an x-y plane and equally dividing the width of the pad or component, when the pad is laid out flat on a horizontal surface. A longitudinal axis is perpendicular to a lateral axis.

“x-y plane,” with reference to an absorbent article, such as a feminine hygiene pad, or component thereof, when laid out flat on a horizontal surface, means any horizontal plane occupied by the horizontal surface or any layer of the article or component.

“z-direction,” with reference to an absorbent article, such as a feminine hygiene pad or component thereof, when laid out flat on a horizontal surface, is a direction perpendicular/orthogonal to the x-y plane.

The terms “top,” “bottom,” “upper,” “lower,” “over,” “under,” “beneath,” “superadjacent,” “subjacent,” and similar terms relating to relative vertical positioning, when used herein to refer to layers, components or other features of an absorbent article such as a feminine hygiene pad, are relative the z-direction and are to be interpreted with respect to the pad as it would appear when laid out flat on a horizontal surface, with its wearer-facing surface oriented upward and outward-facing surface oriented downward.

With respect to an absorbent article such as a feminine hygiene pad, or a component or structure thereof, “wearer-facing” is a relative locational term referring to a feature of the component or structure that when in use that lies closer to the wearer than another feature of the component or structure. For example, a topsheet has a wearer-facing surface that lies closer to the wearer than the opposite, outward-facing surface of the topsheet.

With respect to an absorbent article such as a feminine hygiene pad, or a component or structure thereof, “outward-facing” is a relative locational term referring to a feature of the component or structure that when in use that lies farther from the wearer than another feature of the component or structure. For example, a topsheet has an outward-facing surface that lies farther from the wearer than the opposite, wearer-facing surface of the topsheet.

“Machine Direction” or “MD” as used herein with respect to an absorbent article such as a feminine hygiene pad or component thereof, refers to a direction parallel to the flow of the article or component through processing/manufacturing equipment.

“Cross Machine Direction” or “CD” as used herein with respect to an absorbent article such as a feminine hygiene pad or component thereof, refers to a direction perpendicular/orthogonal to the machine direction.

“Predominant,” and forms thereof, when used to characterize a quantity of weight, volume, surface area, etc., of an absorbent article or component thereof, constituted by a composition, material, feature, etc., means that a majority of such weight, volume, surface area, etc., of the absorbent article or component thereof is constituted by the composition, material, feature, etc.

### General—Absorbent Article; Feminine Hygiene Pad

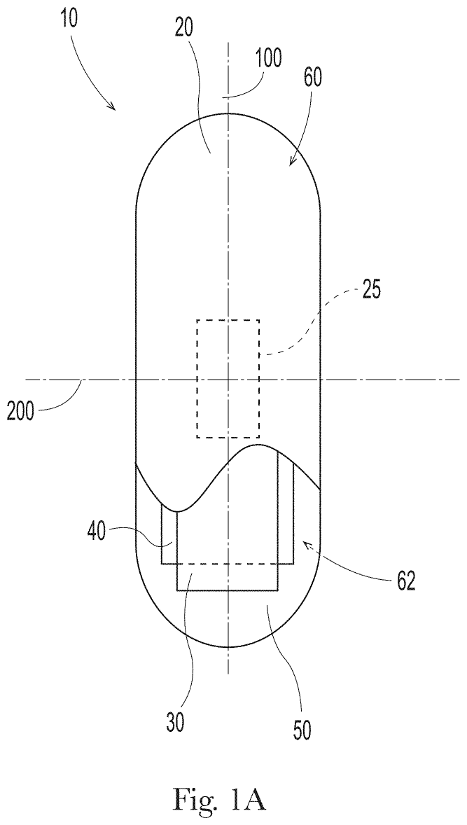

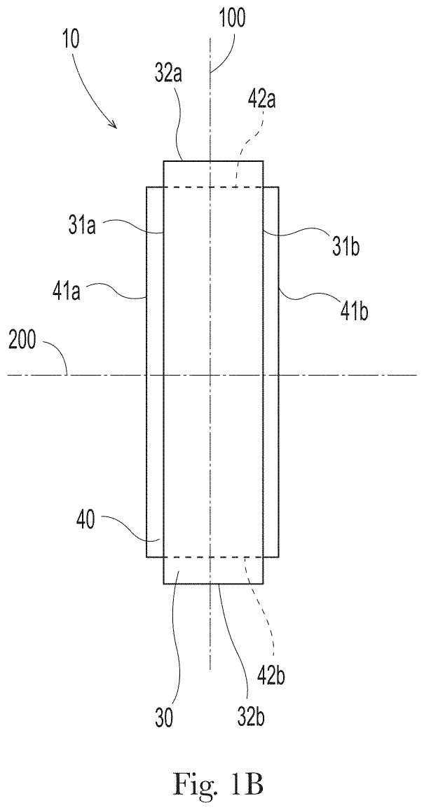

Referring to FIG. 1A, an absorbent article as contemplated herein, such as a feminine hygiene pad 10, will include a wearer-facing surface and an opposing outward-facing surface. A liquid permeable topsheet 20 may form at least a portion of the wearer-facing surface and a liquid impermeable backsheet may form at least a portion of the outward-facing surface. An absorbent core including an absorbent structure 40 is disposed between the topsheet and the backsheet, and a fluid management layer 30 may be included and disposed between the absorbent structure 40 and the topsheet 20. (A fluid management layer as described herein is sometimes known in the art as an “acquisition/distribution layer” “distribution layer” or “secondary topsheet”, whose purpose is to dissipate energy from a fluid gush to the extent needed, provide a temporary volume of space for discharged fluid to occupy during the time required for an underlying absorbent structure to imbibe and absorb the fluid, and to distribute the fluid across the absorbent structure to maximize effective use thereof.) Non-limiting examples of absorbent articles sharing these features include feminine hygiene pads (also known as “sanitary napkins”, “menstrual pads,” etc.), disposable incontinence pads, disposable incontinence underwear, disposable baby diapers and disposable baby/child training pants.

The topsheet 20 and the backsheet 50 may be joined together to form and define an outer periphery of the pad 10. The absorbent structure 40 and the fluid management layer 30 will each be sized to have outer perimeters disposed laterally and longitudinally inboard of the outer periphery. The absorbent structure 40 and the fluid management layer 30 may be dimensioned and shaped substantially similarly or identically to each other in the x-y directions, or they may have respective differing x-y dimensions and/or shapes. One or both may be manufactured to have a rectangular shape as suggested in FIG. 1A, or one or both may be manufactured to have any other suitable shape, such as an oval shape, stadium shape, rounded rectangle shape, hourglass shape, peanut shape, etc. Shapes having concave profiles along the longitudinal edges may in some examples provide for enhanced comfort and/or conformity with the wearer's body.

The topsheet 20 may be joined to the backsheet 50 by attachment any suitable attachment mechanism. The topsheet 20 and the backsheet 50 may be joined directly to each other in the article periphery, and may be indirectly joined together by directly joining them to the absorbent structure 40, the fluid management layer 30, and/or additional layers disposed between the topsheet 20 and the backsheet 50. This indirect or direct joining may be accomplished by any suitable attachment mechanism known in the art. Non-limiting examples of attachment mechanisms may include e.g., fusion bonds, ultrasonic bonds, pressure bonds, adhesive bonds, or any suitable combinations thereof.

Topsheet

### General

Generally it is desirable that the topsheet 20 be compliant, soft feeling, and non-irritating to the wearer's skin. Suitable topsheet materials include a liquid pervious material that is oriented towards and contacts the body of the wearer permitting bodily discharges to rapidly penetrate through it without allowing fluid to flow back through the topsheet to the skin of the wearer. The topsheet, while being capable of allowing rapid transfer of fluid through it, may also provide for the transfer or migration of a lotion composition onto an external or internal portion of a wearer's skin. The topsheet may comprise a nonwoven material.

Nonwoven fibrous topsheets 20 may be produced by any known procedure for making nonwoven webs, nonlimiting examples of which include spunbond processes, carding, wet-laid, air-laid, meltblowing processes, needle-punching, mechanical entangling, thermo-mechanical entangling, and hydroentangling.

Nonwoven materials suitable for use as a topsheet may include one strata of fibers or may be laminate of multiple nonwoven strata, which may comprise the same or different compositions (e.g., spunbond-meltblown laminate). In one specific example, the topsheet is a carded, air-through bonded nonwoven.

Topsheets contemplated herein do not include any predominant fraction of topsheet x-y surface area occupied by film. Some currently known topsheets for feminine hygiene pads include an apertured film, such as a hydroformed film or vacuum-formed film, alone or in combination with an adjacently-disposed nonwoven web material. The film may help to prevent liquids from resurfacing and contacting the wearer. The inventors have found, however, that a topsheet having the features described herein, particularly in combination with the fluid management layer described herein, can effectively prevent rewet to a comparable degree or better, than pads having topsheets comprising film across a predominant portion of topsheet x-y surface area. Without being bound by theory, it is believed that the careful selection of the fiber types in each of the strata in the fluid management layer, and the linear densities of the fiber types, can result in a desired combination of suitably low fluid acquisition time, and low rewet, overcoming the typical tradeoff in these conflicting objectives associated with prior nonwoven topsheets. The improved performance is evident from the new combination of the unique nonwoven topsheet with a fluid management layer of the present disclosure.

### Basis Weight

The topsheet nonwoven may be manufactured to a basis weight of at least about 15 gsm, more preferably at least about 40 gsm, or most preferably at least about 60 gsm, specifically reciting all values within these ranges and any ranges created thereby. In some examples, a nonwoven topsheet contemplated herein may be manufactured to have a basis weight of about 15 gsm to 80 gsm, more preferably about 20 gsm to 60 gsm, or most preferably about 20 gsm to 40 gsm, specifically reciting all values within these ranges and any ranges created thereby. In particular examples the topsheet nonwoven may be manufactured to a basis weight of about 18 gsm to 40 gsm, more preferably about 20 gsm to 30 gsm, even more preferably about 22 gsm to 26 gsm, specifically reciting all values within these ranges and any ranges created thereby. The range of desirable basis weight is influenced, at the lower end of the range, by the need for a level of web tensile strength needed for processing, and by consumer preferences for a level of opacity and substantiality of loft, feel and appearance. The range of desirable basis weight is influenced, at the upper end of the range, by the need for suitable rapid fluid acquisition and passage of fluid through the topsheet, and material cost concerns.

### Fiber Composition

Nonlimiting examples of woven and nonwoven materials suitable for use as the topsheet include fibrous materials made from natural fibers, e.g., cotton, including 100 percent organic cotton, modified natural fibers, semi-synthetic fibers (e.g., fibers spun from regenerated cellulose) synthetic fibers (e.g., fibers spun from polymer resin(s)), or combinations thereof. Synthetic fibers may include fibers spun from single polymers or blends of polymers. Synthetic fibers may include monocomponent fibers, bicomponent fibers or multicomponent fibers. (Herein, bi- or multicomponent fibers are fibers having cross sections divided into distinctly identifiable component sections each formed of a single polymer or single homogeneous polymer blend, distinct from that of the other section(s). Such fibers and processes for making them are known in the art. Examples of bicomponent fiber configurations with substantially round cross sections include side-by-side or “pie slice” configurations, eccentric sheath-core configurations and concentric sheath-core configurations.

Nonwoven topsheets contemplated herein may include fibers having myriad combinations of constituent chemistries. For example, fibers may be spun from polymeric materials, such as polyethylene (PE) and/or polyethylene terephthalate (PET). Fibers may be spun in the form of bi-component fibers. In some examples, bi-component fibers may have a core component of a first polymer (for example, PET) in combination with another polymer as a sheath component, in a sheath-core bicomponent configuration. In more particular examples, PE may form the sheath component in combination with a PET core component. Fibers that include a PET component may be selected to help provide bulk and resilience and a resulting cushiony feel to the nonwoven web. Additionally, fibers that include a PET component, having resilience, help the web retain the area and dimensions of apertures created therethrough, if included.

Other polymeric materials may be included. For example, fibers spun of polypropylene, polyethylene, co-polyethylene terephthalate, co-polypropylene, and other thermoplastic resins may be included. It may be desired that the polymer with the lower melting temperature form the sheath component where sheath-core bi-component fibers are included. Additionally, without wishing to be bound by theory, it is believed that the use of polyethylene terephthalate as a core can help impart resilience to the topsheet.

Polyethylene, as a polymer component from which fibers may be spun, has a relatively lower melting temperature, and exhibits a relatively slick/silky surface feel as compared with other potentially useful polymers. PET has a relatively higher melting temperature, and exhibits relatively greater stiffness and resiliency. Accordingly, in some examples topsheet nonwoven fibers that are of a sheath-core bicomponent configuration may be desired, in which the sheath component is predominantly polyethylene and the core component is predominantly PET. The polyethylene is useful for imparting the fibers and thus the topsheet with a silky feel, and for enabling inter-fiber bonding via heat treatment that cause sheaths of adjacent/contacting fibers to melt and fuse at the lower melting temperature of the polyethylene, while the PET is useful for imparting resilience, and does not melt in the heat treatment process. The inventors have found that a suitable weight ratio in such PE/PET sheath-core bicomponent fibers may be about 40:60 to about 60:40.

### Surface Treatment (Hydrophilicity/Hydrophobicity)

Depending upon the chemical composition thereof, surfaces of fibers will be, inherently, either hydrophilic or hydrophobic. For example, surfaces of fibers spun or otherwise formed from some types of polymers such as polyethylene and polypropylene will be, inherently, hydrophobic. In contrast, surfaces of other types of fibers such as fibers spun from regenerated cellulose (e.g., rayon, viscose, lyocell, etc.) are inherently hydrophilic. Surfaces of natural fibers may be inherently hydrophilic or hydrophobic, but this may depend upon the processing the fibers have undergone. For example, cotton fibers as harvested bear coatings of natural oils and/or waxes and as such their surfaces are hydrophobic. After they have undergone processes including scouring and bleaching, however, the oils and/or waxes will have been stripped away, rendering the fiber surfaces hydrophilic.

Manufacturers and/or suppliers of spun synthetic staple fibers currently apply coatings, in the form of surface finishing agents or processing aids, to the fibers, for purposes of providing lubricity in, e.g., carding processes. These surface finishing agents or processing aids may be formulated to be either hydrophobic or hydrophilic, and substantially durable for purposes herein, in that they will not dissolve in aqueous fluids over the ordinary duration of wear of an absorbent article. Thus, a manufacturer or supplier of spun synthetic staple fibers may offer fibers with either hydrophobic or hydrophilic surface finishes, and currently, several manufacturers in the nonwovens materials industry do this.

Noting that spun synthetic staple fibers may be obtained with either inherently hydrophobic or hydrophilic surfaces, or obtained with surface finishes that render their surfaces hydrophilic or hydrophobic at the purchaser's option, it may be desirable to choose fibers with surfaces that are either hydrophilic (“hydrophilic fibers”) or hydrophobic (“hydrophobic fibers”), or choose a blend of fibers of both types.

In some examples it may be preferable that the fiber constituents of the topsheet be, by weight, predominantly, substantially, or entirely hydrophobic, or rendered hydrophobic via fiber surface finish. A topsheet formed of a nonwoven web with predominately hydrophobic fiber constituents will be resistive to rewetting. On the other hand, if the sizes of the pores or inter-fiber voids within the fibrous structure of such nonwoven web are not sufficiently large, the topsheet may resist the passage of fluid from the wearing facing surface through to the absorbent core components of the article therebeneath, i.e., will not readily/rapidly acquire fluid, unless other features are included in combination, as described herein.

In other examples, fibers constituting portions, a majority (by surface area), or all, of the section of web material from which of the topsheet is formed, may be a blend of both hydrophobic fibers and hydrophilic fibers. In such examples, the hydrophilic fibers can serve to help wick fluid from the wearer-facing surface of the topsheet down to the absorbent core components beneath, while the hydrophobic fibers can serve to help the topsheet resist rewetting. The inventors have discovered that a successful balance may be struck for such examples.

Accordingly, in some examples the topsheet nonwoven may include a mix of hydrophobic and hydrophilic fibers. For example, the nonwoven may include at least about 40 percent, more preferably at least about 50 percent, or most preferably at least about 60 percent hydrophilic fibers by weight of the fibers, specifically including all values within these ranges and any ranges created thereby. In more particular examples, the nonwoven topsheet may comprise about 40 percent to 70 percent, more preferably about 45 percent to 68 percent, or most preferably from about 50 percent to 65 percent, by weight, hydrophilic fibers, specifically reciting all values within these ranges and any ranges created thereby. The topsheet nonwoven may include a blend of hydrophilic fibers and hydrophobic fibers in a weight ratio of hydrophilic fibers to hydrophobic fibers of 30:70 to 70:30, more preferably 35:65 to 65:35, and even more preferably 40:60 to 60:40. As noted above, the hydrophilicity of the hydrophilic fibers may be effected by application of a surface treatment composition.

Without wishing to be bound by theory, it is believed that where a majority of the fibers are hydrophilic, fluid acquisition speed can be improved by combination with other features described herein, while not overly impacting rewet in a negative or unacceptably negative manner. Where less rewet is the goal, then the converse may be true. In this circumstance, a higher weight fraction of hydrophobic fibers may be desired.

### Linear Density

Fibers are typically manufactured, selected and purchased by linear density specification, such expressed as denier or decitex. For fibers of a given polymer constitution, linear density correlates with fiber size/diameter.

The fibers constituting the topsheet may selected to have an average linear density of about 1.0 to 3.0 denier, more preferably about 1.5 to 2.5 denier, and even more preferably about 1.8 to 2.2 denier, and all combinations of subranges within these ranges are contemplated herein. Fibers with varying linear densities within the ranges set forth above may be selected and included as well.

Without wishing to be bound by theory, it is believed that, for a nonwoven of particular selected basis as contemplated herein, inclusion of fibers having a linear density greater than about 3.0 denier may result in a topsheet that lacks, for some consumers, a sufficiently soft feel, since such relatively larger fibers would tend to be stiffer. Conversely, a selection of fibers having a linear density less than about 1.0 denier result in unduly small interstitial spaces/voids between and among the fibers, and make fluid acquisition and movement through the topsheet more difficult.

### Staple Fiber Length

Suitable fibers may be staple fibers having a length of at least about 30 mm, 40 mm, or 50 mm, up to about 55 mm, or about 30 to 55 mm, or about 35 to 52 mm, reciting for said range every 1 mm increment therein. In particular example, staple fibers may have a length of about 38 mm.

### Apertures

The inventors have found that, in topsheet nonwovens that are formed of fibers of relatively small size/linear density and/or fibers that are predominantly, substantially or entirely hydrophobic, acquisition speed may be substantially increased by forming a pattern of apertures through the web. Generally, the preferred apertures will have sizes that are substantially larger than the average pore/void size within the nonwoven web. The apertures may be formed by any suitable, known pin punching process. The process may include use of pins arranged in any desired pattern and radially extending from a pinned cylindrical roller, coupled with a mating cylindrical roller having pin receiving holes in its surface. One or both rollers may be heated to a temperature sufficient to cause softening and plastic deformation of the nonwoven web fibers, without melting them. Passage of the nonwoven web material through the nip between these rollers can effect enduring or substantially permanent displacement of the positions of the fibers along x- and y-directions, as well as the z-direction, within the nonwoven structure, thereby creating apertures through the web that substantially retain their sizes and shapes as the web is manipulated in later/downstream processes such rolling, unrolling and absorbent article manufacturing processes. Preferably, the aperture-forming process follows bonding of the web via heat treatment, to provide for more reliable formation of more dimension- and shape-stable apertures.

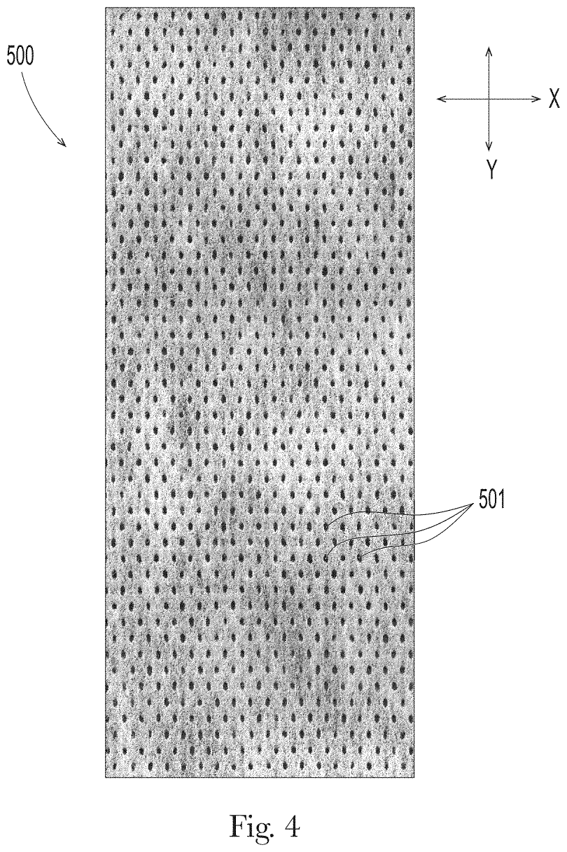

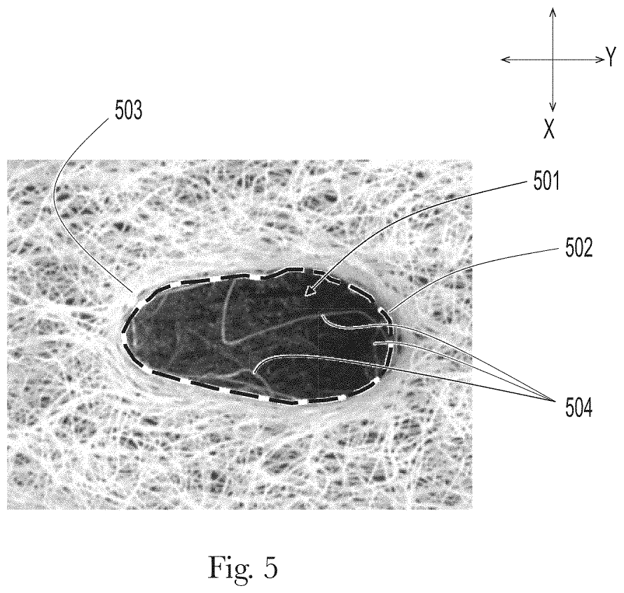

An example of a section of topsheet nonwoven web material 500 having a pattern of apertures 501 therethrough is depicted in FIG. 4. A magnified image of example of an aperture through a nonwoven web material is depicted in FIG. 5. Apertures are distinguishable from randomly-disposed pores or voids through the nonwoven web material, in that they are created by readily discernible displacements of fibers, along x-y directions, resulting in concentrated groups of displaced fibers that define the perimeter of a z-direction opening through the nonwoven web that is relatively larger than the randomly disposes pores or voids between and among the fibers constituting the material. Apertures may be created through the web via a process and equipment configured to impart an average x-y dimension aperture area of 0.5 mm² to 2.5 mm², preferably about 0.6 mm² to 1.2 mm², and all combinations of subranges within these ranges are contemplated herein. Herein, the x-y dimension area of an aperture is defined by visually discernible inside edges of the concentrations of displaced fibers 503 about the perimeter of the aperture. Stray individual fibers that may have escaped the main structure and/or the concentrations of displaced fibers about the perimeter, and cross into or through the main open area of the aperture (by way of illustrative example, stray individual fibers 504 shown in FIG. 5) are not considered subtractive from the aperture area for purposes herein. Further, without wishing to be bound by theory, it is believed that the where the shapes of the apertures are too oblong or narrow, fluid acquisition speed may be negatively impacted. Accordingly, it may be desired that the apertures have a limited maximum average x-y direction aspect ratio (greatest dimension: smallest dimension in x-y directions). Thus, it may be desired that the average that the average aspect ratio of the apertures be about 2.5:1 to 1:2.5, more preferably about 1.5:1 to 1:1.5, or most preferably about 1:1, and all combinations of subranges within these ranges are contemplated herein. Further, it is preferable for purposes of retaining structural integrity of the web and shape integrity of the apertures, that the x-y plane shapes of the majority or all of the apertures in, at least, the region of interest 25 (“ROI,” defined below; see FIG. 1A) if not the majority or entirety of the topsheet, be rounded shapes (e.g., circular, oval, ovoid, elliptical, stadium, etc.), having no sharp corners. Accordingly, it may be desired that the pinned roller used to create the apertures have pins that do not have sharp corners, when viewed along a radially inward direction toward the axis of the roller.

Collectively, the aperture areas of all of the apertures in the portion of interest of the topsheet amount to an open x-y plane area (“open area”) in the topsheet nonwoven. In combination with a desired average aperture size, the inventors have identified a desired open area, in order to effectively mitigate potential obstacles to fluid acquisition that may result from constitution of fibers of finer denier and/or fibers that are predominantly hydrophobic. Accordingly, it may be desired that apertures, if included, collectively provide an open area of 6 percent to 25 percent, more preferably 8 percent to 18 percent, and even more preferably 10 percent to 15 percent, and all combinations of subranges within these ranges are contemplated herein. It is preferred that such amount of open area be present in substantially the entirety of the portion of the topsheet overlying the fluid management layer and/or absorbent structure, or at least, in the region of interest 25 (“ROI”) defined below (and see FIG. 1A). The lower limits of these ranges are imposed by the need for efficacy/performance; the apertures should provide at least a minimum amount of open area in order to be effective as may be included for the purposes described herein. The upper limits of these ranges are imposed by the need for consumer acceptance; if the open area is too great, consumers may perceive that the topsheet is fragile or of poor quality; and further, the topsheet becomes less effective at retaining fluid therebeneath, and at masking staining by absorbed fluid present in the absorbent components beneath the topsheet.

Referring to FIG. 1A, for purposes contemplated herein, a region of interest 25 (“ROI”) is a rectangular section of the topsheet that is 60.0 mm long in the longitudinal direction and 30.0 mm wide in the lateral direction, and is centered at the longitudinal and lateral center, in an x-y plane, of the fluid management layer. The percent fraction open area of the ROI 25 is the fraction of the x-y area therewithin that is open therethrough in the z-direction, by the collective presence of the apertures 501 therewithin. Expressed differently, the percent fraction open area within the ROI is the total x-y area of the apertures within the ROI, divided by 1,800 mm², times 100%.

The percent fraction open area in the ROI may be obtained in some examples from the specifications given to or provided by the manufacturer of the topsheet nonwoven web material. Where this is unavailable, it may be measured via any suitable measurement technique that may applied, in a manner consistent with the description of the x-y dimension area of an aperture area and description of “open area,” above, which may include but is not limited to the Apertures Open Area Measurement Method set forth below.

### Bonding

Generally, it is desirable that the fibers forming the topsheet nonwoven be bonded following the carding/fiber laydown process, to impart a fabric-like structure and tensile strength (in both the MD and the CD) needed for the web to substantially retain its structure in downstream/later processes, and in the form of a topsheet, during use by a user/wearer. As an alternative to other methods of bonding such as mechanical compression spot bonding (with or without application of heating energy), adhesive bonding, etc., it has been found that bonding via air-through heating is effective for creating fiber-to-fiber bonds and imparting structure integrity to the web, while preserving inter-fiber pore/void size and loft, and imparting resiliency, to the nonwoven. In examples of suitable processes, air heated to the selected heating temperature is blown and/or drawn (via vacuum) through the carded fiber web as it is conveyed on a carrier belt along a machine direction, through an oven or heating chamber. When operating parameters including heating air temperature and velocity, and exposure time, are appropriately adjusted, a plurality of randomly distributed fiber-to-fiber bonds may be created within the fiber network, which impart structural integrity to the web. When constituent fibers are, for example, sheath-core bicomponent fibers in which the sheath component is a polymer having a melting temperature lower than that of the core component, the process may be configured such that fusion bonds form between sheaths of adjacent contacting fibers without complete melting and loss of structure of the sheaths, while the cores remain in place, un-melted. In such process, the bonds may be formed without application of compression, and thus, without associated loss of caliper of the web and reduction in size of the inter-fiber pores/voids.

### Applied Anti-Stick Agent

The absorbent article may include an anti-stick agent applied, to at least a portion of the wearer-facing surface of the topsheet, wherein the anti-stick agent includes a polypropylene glycol material. It is believed that an applied anti-stick agent as described herein serves functions that include reducing adherence of menstrual fluid to the user/wearer's skin, and facilitation of migration of menstrual fluid from the wearer-facing surface of the topsheet, down therethrough to the fluid management and/or absorbent structure layers beneath. Serving these functions can enhance user/wearer perceptions of cleanliness of her skin and of the topsheet, especially after repeated discharges of menstrual fluid. Examples of a suitable anti-stick agents and/or surfactants useful therein are disclosed in US 2009/0221978 (wherein the composition is called a “lotion”) and U.S. Pat. No. 8,178,748.

The anti-stick agent may include a polypropylene glycol (“PPG”) material. In some examples, the anti-stick agent may consist essentially of a polypropylene glycol material, preferably a polypropylene glycol homopolymer such as polypropylene glycol, and optionally, a carrier. In other examples, the anti-stick agent may include a polypropylene glycol material selected from the group consisting of polypropylene glycol copolymer, polypropylene glycol surfactant, and mixtures thereof. The anti-stick agent including the polypropylene glycol material may serve to help reduce the adherence of menstrual fluid to the topsheet, and upon contact transfer of anti-stick agent to the user/wearer, reduce the adherence of fluid to her skin, thereby reducing staining on the topsheet and reducing soiling of the skin. The anti-stick agent may also help to improve continuous fluid acquisition of the absorbent article.

The anti-stick agent may be applied in any known manner, in any known pattern, and to the wearer-facing surface of the topsheet 20. For example, the anti-stick agent may be applied in a pattern of generally parallel, longitudinally- or laterally-oriented stripes or bands. To avoid compressing or displacing any portion of the topsheet nonwoven or any three-dimensional features thereof, it may be desired that the anti-stick agent be applied via spraying. A substantially uniformly sprayed application may be preferred.

The quantity of anti-stick agent applied may vary, and can be adjusted for specific needs. For example, while not being bound by theory, it is believed that anti-stick agent may be applied at levels that are effective, of at least about 0.1 gsm, 0.5 gsm, 1 gsm, 2 gsm, 3 gsm, 4 gsm, 5 gsm, 10 gsm, up to about 15 gsm, or up to about 12 gsm, or up to about 10 gsm. It is believed that efficacy is not further enhanced above these upper limits, and so applications at basis weights exceeding these upper limits may be needless usage (waste) of anti-stick agent. The anti-stick agent can be applied within any subrange defined by any of the levels recited above (e.g. from about 0.1 gsm to about 15 gsm). These levels refer to the area of the topsheet surface to which the anti-stick agent is actually applied. It may be preferred that a majority, substantially all, or all of the surface area of the topsheet overlying the fluid management layer and/or absorbent core have the anti-stick agent applied. This is because, as is believed, the anti-stick agent may enhance the ability of the topsheet to resist rewetting.

The anti-stick agent contemplated herein offers significant advantages over other anti-stick agents, including non-PPG derived surfactants and other surface modifying agents. The advantages may be deemed particularly useful for feminine hygiene pads. Without intending to be bound by theory, it is believed that the superior fluid handling properties of the PPG materials identified herein is a result of the way in which the PPG materials act on the solid components of menstrual fluid, as opposed to surface energy treatments which act on the water component of menses. Surface energy treatments may be less effective due to the presence of polar and dispersive components in menstrual fluid, which may inhibit the effectiveness of surface energy treatments. Because the PPG materials identified herein are typically not readily soluble in menstrual fluid, they can effectively coat surfaces without dissolving in the fluid, which provides a hydrated barrier whose electron donating dipoles repel negatively dipoled proteins, thereby rendering the menstrual fluid less apt to adhere to surfaces of the article or the wearer's skin. Less adherence of menstrual fluid to the wearer's skin and/or to the topsheet promotes better and faster fluid movement through the topsheet, and fewer, smaller and/or less visible stain patterns on used products.

The PPG materials identified herein can be applied as one component in an anti-stick agent, or can be applied neat (i.e., the anti-stick agent consists of PPG material). PPG materials, either neat and/or as part of an anti-stick agent, can be applied at varying quantity levels, depending on the fluid handling properties desired and desired treatment of the wearer's skin. PPG materials may be applied to the outer surface of the topsheet in any pattern, such as full coat, stripes or bands (oriented in the MD or CD direction), droplets, spiral patterns, and other patterns. An anti-stick agent including the PPG material may also be disposed near channels or embossed areas, when present.

The anti-stick agent contemplated herein may include a PPG material. PPG materials suitable for purposes contemplated herein include PPG homopolymer materials, PPG copolymer materials, and PPG surfactant materials, as well as mixtures thereof. The anti-stick agent may further comprise other optional ingredients. In one embodiment, the anti-stick agent consists essentially of, or consists of, a PPG material, preferably polypropylene glycol. In another embodiment, the anti-stick agent comprises a PPG material selected from the group consisting of polypropylene glycol copolymer, polypropylene glycol surfactant, and mixtures thereof.

The anti-stick agents contemplated herein may include a PPG material at a level of about 0.1% to 100%, by weight of the anti-stick agent. In some examples, the anti-stick agent may include less than about 10%, preferably from about 0.5% to 8%, and more preferably from about 1% to 5%, of a PPG material, by weight of the anti-stick agent. In other examples, the anti-stick agent may include at least about 50%, preferably about 75% to 100%, and more preferably about 90% to 100%, of a PPG material, by weight of the anti-stick agent.

Suitable PPG homopolymer materials may include those corresponding to the following formula:

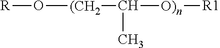

wherein R is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and wherein R is preferably hydrogen or methyl;
wherein R1 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and wherein R1 is preferably hydrogen or methyl; and
wherein n is from 3 to 160, preferably from 5 to 120, more preferably from 10 to 100, and more preferably from 20 to 80.

Optionally, the PPG homopolymer may include low level of glycerol or butanediol as part of its monomer raw material. If included, the preferred ratio of glycerol or butanediol to propylene glycol may be 1:1000 to 1:2, most preferably 1:100 to 1:5. The PPG homopolymer may have, but is not necessarily limited to, CAS Numbers 25322-69-4, 25791-96-2 and 25231-21-4, wherein the latter is most preferred.

Non-limiting examples of suitable PPG homopolymer materials include polypropylene glycol 4000 such as Pluriol P-4000 (BASF), Alkapol PPG-4000 (Alkaril Chemical) and Niax Polyol PPG 4025 (Union Carbide); polypropylene glycol 3500; polypropylene glycol 3000 such as Niax PPG 3025 (Union Carbide); polypropylene glycol 2000 such as Alkanol PPG-2000 (Alkaril Chemical) and Pluriol P-2000 (BASF), polypropylene glycol 1200 such as Alkapol PPG-1200 (Alkaril Chemical) and Glucam P-20 Humectant (Noveon); polypropylene glycol 1000 such as Niax PPG 1025 (Union Carbide); polypropylene glycol 600 such as Alkanol PPG-600 (Alkaril Chemical) and Glucam P-10 Humectant (Noveon); polypropylene glycol 400 such as Alkanol PPG-425 (Alkaril Chemical). polypropylene glycol 4000 glycerol ether such as Pluriol T-4000 (BASF); polypropylene glycol 2000 glycerol ether, polypropylene glycol 2000 butanediol ether, polypropylene glycol 1500 glycerol ether such as Pluriol T-1500 (BASF), polypolypropylene glycol 4000 with monomethyl ether, polypropylene glycol 2000 with dimethyl ether, polypropylene glycol 4000 with monobutyl ether, polypropylene glycol 2000 with monobuytyl ether, polypropylene glycol 1200 with dibutyl ether, polypropylene glycol 4000 with bis(2-aminopropyl ether), PPG-10 methyl glucose ether and PPG-20 methyl glucose either.

Suitable PPG homopolymer materials will typically have a number average molecular weight of about 400 to 10,000, preferably about 600 to 6,000, and more preferably about 1,200 to 4,800.

Suitable PPG copolymer materials include those in which the polyprolyene glycol segments are present as an internal block component and/or as a terminal component, of the copolymer structure. The following formulae illustrate the internal block components and terminal block components:

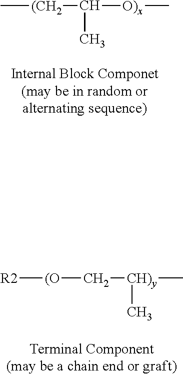

wherein x is 2 to 120, preferably 2 to 80, and more preferably 3 to 60; y is 2 to 100, preferably 2 to 50, and more preferably 3 to 30; R2 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate, and wherein R2 is preferably hydrogen, methyl, ethyl, isopropyl or isobutyl.

Polymers suitable to form propoxylated copolymers with PPG for the present anti-stick agents include homopolymers of alkyl methicone, phenyl methicone, dimethicone, alkyl trimethicone, phenyl trimethicone, polyol, polyether (e.g., polyoxymethylene, polyoxyethylene and polyoxypropylene), polyimine, polyamide, polyacrylate, polyester, and copolymers containing one or multiple of these polymeric units. Non-limiting examples of suitable polymers include polydimethyl siloxane, polyethylimine, polyacrylic acid, poly(ethylene-co-acrylic acid), polymethacrylic acid, poly(ethylene-co-methacrylic acid), poly(vinyl acetate), polyvinylpyrrolidone, poly(ethylene-co-vinyl acetate), poly(butanediol), poly(neopentyl glycol), poly(ethylene adipate), poly(butylene adipate), poly(ethylene glutamate), poly(butylene glutamate), poly(ethylene sebacate), poly(butylene sebacate), poly(ethylene succinate), poly(butylene succinate), poly(ethylene terephthalate), poly(butylene terephthalate), polycaprolactone, and polyglycerol.

Non-limiting examples of suitable PPG copolymer materials include PPG-12 dimethicone such as Sisoft 910 (Momentive); bis-PPG-15 dimethicon/IPDI copolymer such as Polyderm-PPI-SI-WI (Alzo); PPG/polycaprolactone block copolymer; PPG/polybutanediol/PEG triblock copolymer; polyethylimine/PPG copolymer and polyacrylic acid-g-PPG graft copolymer.

Another suitable PPG material includes PPG surfactant materials. The following formula represents suitable PPG surfactant materials wherein the PPG segments constitute a part of the head functional group:

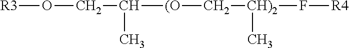

wherein R3 is hydrogen, alkyl, alkyl carbonyl, alkylenelamine, alkylenelamide, alkylene phosphate, alkylene carboxylic acid, alkylene sulfonate salt and alkylene quat with the maximum number of carbon element less than or equal to 6; R4 is octyl, nonyl, decyl, iosdecyl, lauryl, myristyl, cetyl, isohexadecyl, oleyl, stearyl, isostearyl, tallowoyl, linoleyl, jojoba, lanolin, behenyl, C24-C28 alkyl, C30-C45 alkyl, dinonylphenyl, dodecyl phenyl, or soya; z is from 1 to 100, preferably from 2 to 30, and more preferably from 3 to 25; and F is a functional group selected from the group consisting of ether groups (including oxy, glyceryl, glucose, sorbitan, sucrose, monoethanolamine or diethanolamine), ester groups (including ester, glyceryl ester, glucose ester, sorbitan ester or sucrose ester), amine groups, amide groups, and phosphate ester groups.

The following formula represents suitable PPG surfactant materials wherein the PPG segments constitute an internal block group:

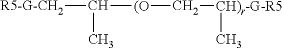

wherein R5 is hexyl, 2-ethylhexyl, octyl, nonyl, decyl, isodecyl, lauryl, cocoyl, myristyl, cetyl, isohexadecyl, oleyl, stearyl, isostearyl, tallow, linoleyl, octyl phenyl, or nonyl phenyl; r is from 1 to 120, preferably from 4 to 50, and more preferably from 6 to 30; and G is ether, ester, amine, or amide linkage.

Non-limiting examples of suitable PPG surfactant materials include PPG-30 cetyl ether such as Hetoxol C3OP (Global Seven); PPG-20 methyl glucose ether distearate such as Glucam P-20 Distearate Emollient (Noveon), PPG-20 methyl glucose ether acetate, PPG-20 sorbitan tristearate, PPG-20 methyl glucose ether distearate, PPG-20 distearate, PPG-15 stearyl ether such as Alamol-E (Croda-Uniqema) and Procetyl 15 (Croda), PPG-15 stearyl ether benzoate, PPG-15 isohexadecyl ether, PPG-15 stearate, PPG-15 dicocoate, PPG-12 dilaurate, PPG-11 stearyl ether such as Varonic APS (Evonik); PPG-10 cetyl ether such as Procetyl 10 (Croda); PPG-10 glyceryl stearate, PPG-10 sorbitan monosterate, PPG-10 hydrogenated castor oil, PPG-10 cetyl phosphate, PPG-10 tallow amine, PPG-10 oleamide, PPG-10 cetyl ether phosphate, PPG-10 dinonylphenolate, PPG-9 laurate, PPG-8 dioctate, PPG-8 diethylhexylate, PPG-7 lauryl ether, PPG-5 lanolin wax ether, PPG-5 sucrose cocoate, PPG-5 lanolin wax, PPG-4 jojoba alcohol ether, PPG-4 lauryl ether, PPG-3 myristyl ether such as Promyristyl PM-3 (Croda), PPG-3 myristyl ether propionate such as Crodamol PMP (Croda), PPG-3 benzyl ether myristate such as Crodamol STS (Croda), PPG-3 hydrogenated castor oil such as Hetester HCP (Alzo), PPG-3-hydroxyethyl soyamide, PPG-2 Cocamide, PPG-2 lanolin alcohol ether and PPG-1 coconut fatty acid isopropanolamide such as Amizett IPC (Kawaken Fine Chemicals). A particularly preferred example of a suitable PPG material is PPG-15 stearyl ether, such as the product sold as CETIOL E, by BASF Corporation (Florham Park, N.J., USA) and/or BASF SE (Ludwigshafen, Germany).

The anti-stick agents contemplated herein may include the carrier at a total carrier concentration ranging from about 60% to 99.9%, preferably about 70% to 99.5%, more preferably about 80% to 99% by weight of the anti-stick agent.

Carriers suitable herein may include oils or fats such as natural oils or fats, or natural oil or fat derivatives, in particular of plant or animal origin. Non-limiting examples include avocado oil, apricot oil, apricot kernel oil, babassu oil, borage oil, borage seed oil, calendula oil, camellia oil, canola oil, carrot oil, cashew nut oil, castor oil, chamomile oil, cherry pit oil, chia oil, coconut oil, cod liver oil, corn oil, corn germ oil, cottonseed oil, eucalyptus oil, evening primrose oil, grape seed oil, hazelnut oil, jojoba oil, juniper oil, kernel oil, linseed oil, macadamia oil, meadowfoam seed oil, menhaden oil, mink oil, moringa oil, mortierella oil, olive oil, palm oil, palm kernel oil, peanut oil, peach kernel oil, rapeseed oil, rose hip oil, safflower oil, sandlewood oil, sesame oil, soybean oil, sunflower oil, sunflower seed oil, sweet almond oil, tall oil, tea tree oil, turnip seed oil, walnut oil, wheat germ oil, zadoary oil, or the hardened derivatives thereof. Hardened oils or fats from vegetal origin can include, e.g., hardened castor oil, peanut oil, soya oil, turnip seed oil, cottonseed oil, sunflower oil, palm oil, kernel oil, linseed oil, corn oil, olive oil, sesame oil, cocoa butter, shea butter and coconut oil.

Other non-limiting examples of fats and oils may include: butter, C12-C18 acid triglyceride, camellia oil, caprylic/capric/lauric triglyceride, caprylic/capric/linoleic triglyceride, caprylic/capric/stearic triglyceride, caprylic/capric triglyceride, cocoa butter, C10-C18 triglycerides, egg oil, epoxidized soybean oil, glyceryl triacetyl hydroxystearate, glyceryl triacetyl ricinoleate, glycosphingolipids, human placental lipids, hybrid safflower oil, hybrid sunflower seed oil, hydrogenated castor oil, hydrogenated castor oil laurate, hydrogenated coconut oil, hydrogenated cottonseed oil, hydrogenated C12-C18 triglycerides, hydrogenated fish oil, hydrogenated lard, hydrogenated menhaden oil, hydrogenated mink oil, hydrogenated orange roughy oil, hydrogenated palm kernel oil, hydrogenated palm oil, hydrogenated peanut oil, hydrogenated shark liver oil, hydrogenated soybean oil, hydrogenated tallow, hydrogenated vegetable oil, lanolin and lanolin derivatives, lanolin alcohol, lard, lauric/palmitic/oleic triglyceride, lesquerella oil, maleated soybean oil, meadowfoam oil, neatsfoot oil, oleic/linoleic triglyceride, oleic/palmitic/lauric/myristic/linoleic triglyceride, oleostearine, olive husk oil, omental lipids, orange roughy oil, pengawar djambi oil, pentadesma butter, phospholipids, pistachio nut oil, placental lipids, rapeseed oil, rice bran oil, shark liver oil, shea butter, sphingolipids, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, vegetable oil, wheat bran lipids, and the like, as well as mixtures thereof. A particularly preferred example of a suitable carrier is caprylic/capric triglyceride. This material is currently available as, e.g., MYRITOL 318, a product of BASF Corporation (Florham Park, N.J., USA) and/or BASF SE (Ludwigshafen, Germany).

Other suitable carriers may include mono- or di-glycerides, such as those derived from saturated or unsaturated, linear or branch chained, substituted or unsubstituted fatty acids or fatty acid mixtures. Examples of mono- or diglycerides include mono- or di-C12-24fatty acid glycerides, specifically mono- or di-C16-20fatty acid glycerides, for example glyceryl monostearate, glyceryl distearate.

Carriers can also include esters of linear C6-C22-fatty acids with branched alcohols.

Carriers contemplated herein may also include sterols, phytosterols, and sterol derivatives. Sterols and sterol derivatives that can be used in the anti-stick agents of the invention include, but are not limited to: (3-sterols having a tail on the 17 position and having no polar groups for example, cholesterol, sitosterol, stigmasterol, and ergosterol, as well as, C10-C30 cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, “AVOCADIN” (trade name of Croda Ltd of Parsippany, N.J.), sterol esters and similar compounds, as well as mixtures thereof. A commercially available example of phytosterol is GENEROL 122 N PRL refined soy sterol from Cognis Corporation of Cincinnati, Ohio.

Absorbent Core

### Fluid Management Layer

The absorbent core may include a fluid management layer 30.

A fluid management layer as contemplated herein may include a structured accumulation of carded, integrated fibers. The fluid management layer adds caliper to the absorbent article and is typically compressible, and may be composed and structured to be resilient, which can impart a feeling of softness and/or a “cushiony” feel to the article. Often there is a tradeoff between resiliency and softness in configuring layer components for absorbent article. Softer (i.e., more compliant or pliable) materials may have less tendency to recover their shape following deformation resulting from application of force in one or more directions. The converse may be true for resilient materials. Among materials typically included as components of absorbent articles, resilient materials recover their original size and/or shape following deformation resulting from application of force; however, they may not be perceived as feeling “soft.” Additionally, many absorbent articles or layer components thereof can exhibit good resilience properties when dry; however, upon absorption of fluid, their resiliency decreases substantially. The absorbent articles contemplated herein exhibit good resiliency properties both in dry and wet conditions.

In addition to the softness and resiliency benefits of compositions and structures for fluid management layers contemplated herein, stain size control and faster fluid acquisition may be obtained. Stain size is important in the way the absorbent article is perceived by the user. For feminine hygiene pads, when a stain visible on the pad after a duration of use/wear is relatively large along x-y directions, users may perceive that the pad is near failure based on the appearance of the stain and its proximity to the outer periphery of the pad. In contrast, a smaller stain can have a reassuring effect on the user/wearer, by creating a perception that the pad is not near failure because the edges of the stain lie substantially longitudinally and/or laterally short of the outer periphery of the pad.

Fluid acquisition speed of the pad may be deemed important to the user/wearer, as rapid acquisition can help make the user/wearer feel dry and clean. When the pad requires a relatively long time to drain discharged fluid from the topsheet, it can cause the user to feel wetness, and feel unclean.

As noted, the fluid management layer 30 as contemplated herein may be formed of a nonwoven material of integrated, carded fibers. The fluid management layer contemplated herein may include one or more webs of carded fibers, which are integrated with one another. Where only a single carded web is included, the fibers of the single web may be integrated.

Myriad variations of combinations of compositions, manufacturing methods and configurations for a fluid management layer 30 may be designed and manufactured. However, in many circumstances it may be desirable that the fluid management layer have adequate pore volume to allow for rapid acquisition/intake of discharged fluid.

A plurality of carded webs may be included to constitute the fluid management layer, and may be different from one another. For example, one carded web may have differing fiber constituents and/or blends thereof, than one or more of the others. For example, assuming that a first carded web would be closest to the wearer-facing surface in an absorbent article, the fiber constituent selection and manufacturing process for the first carded web may be configured such that the first web has greater pore volume than one or more underlying carded webs of the fluid management layer. An underlying second or intermediate carded web may be included and similarly configured. In contrast, an underlying third or lowermost carded web may be configured to draw fluid from the void space of the first and second carded webs and effectively distribute these liquid insults to/across an underlying absorbent structure. Where a fiber constitution of one of the carded webs constituting the fluid management layer is different than a fiber constitution of another carded web constituting the fluid management layer (whether fibers of the respective carded webs are integrated, or not), the fluid management layer is deemed herein a heterogenous configuration. Alternatively, where the carded web(s) constituting the fluid management layer all have the same fiber constitution, the fluid management layer is deemed herein a homogeneous configuration.

After fibers of a plurality of the carded web(s) constituting a fluid management layer are integrated, the respective carded webs cannot readily be separated. However, each carded web of the fluid management layer forms a stratum in the layer. Each stratum can retain its unique properties for at least a portion thereof along the z-direction, even when fibers thereof are integrated into superadjacent/subjacent carded web(s). The fluid management layer can draw fluid through and from the topsheet via capillary action or wicking forces, of sufficient magnitude to overcome any resistance to passage of the fluid through the topsheet, or attraction the topsheet may have for the fluid, that may be present as a result of the composition and/or configuration of the topsheet. The fluid management layer also can accept and contain a gush of fluid by providing pore volume as a temporary reservoir, together with distribution functions, to efficiently utilize the absorbent structure, give it time to imbibe and absorb the fluid.

Absorbent articles that exhibit a soft cushiony feel, good resiliency and fluid handling characteristics are contemplated herein. Toward imparting these characteristics, the caliper of the fluid management layer may be deemed important. Typical calipers of webs from conventional spunlace lines achieve a caliper factor (caliper per 10 gsm of basis weight) of 0.03 mm/gsm to 0.12 mm/gsm. In contrast, the fluid management layers contemplated herein can exhibit a caliper factor of at least 0.13 mm/gsm, at least about 0.15 mm/gsm, or about 0.2 mm/gsm, including any values within these ranges and any ranges created thereby. The fluid management layers contemplated herein can have a caliper factor of between 0.13 mm/gsm to about 0.3 mm/gsm, or from about 0.14 mm/gsm to about 0.25 mm/gsm, or from about 0.15 mm/gsm to about 0.22 mm/gsm, including all values within these ranges and any ranges created thereby. Caliper data is provided hereafter for Prototype sample 1 and Comparative sample 1. The caliper and caliper factor of the fluid management layers of the present disclosure may be determined by the Caliper and Caliper Factor test methods disclosed herein. It is important to note that the caliper factors mentioned heretofore are with regard to caliper obtained using the Caliper measurement method set forth below.

It has been discovered that, in order to obtain the increase in caliper factor, a comparatively simpler process may be utilized to produce the spunlace web. Typically, the web path through a hydroentangling line is tortuous, and subjects the web to both compressive and tensile stresses. This tortuous web path requires water jet pressures of a magnitude sufficient to entangle the fibers, to the extent needed to impart tensile strength to the web sufficient for it to survive subsequent processing. These water jets are typically directed at both surfaces of the web. The water pressure required to cause sufficient entanglement for web tensile strength is generally greater than the pressure needed to impart the desired fluid handling pore structure, and also causes substantial reduction in the caliper of the resulting hydroentangled/spunlaced web. Additionally, typically, the web will be subject to significant z-direction compression and machine-direction tensile stress as it is routed around a variety of vacuum drums and rolls such that additional water jets can further entangle the constituent fibers of the strata. Further routing around dryer drums subjects the web to them to additional z-direction compressive and machine-direction tensile forces.

The inventors have discovered, however, that through the use of a simplified web path that reduces radial compressive stresses/excessive tensile forces and the appropriate selection of fibers in the fluid management layer, caliper of the web from which the fluid management layer is made can be retained. Use of rolls and the number of water jets utilized can be reduced to simplify the process. Although the resulting extent of fiber entanglement is not as great as that provided by conventional processes sufficient tensile strength can be imparted to the web via selection of the appropriate combination of fibers as disclosed herein, e.g., stiffening fibers which can be fusion bonded via a heat treatment process which causes fusion bonding between them where they contact each other. The simplified path and appropriate fiber selection as described herein, allow the manufacturer to impart fluid management layer web material(s) with a caliper factor that have heretofore not been attainable.

Significantly, the caliper factors for samples of prototype fluid management layer materials manufactured as contemplated herein were derived from caliper data for prototype material which had been wound in rolls for storage and shipping. It is believed that caliper measurements of the same materials could be taken prior to such winding, which would result in even higher caliper factors. However, such pre-winding caliper measurements might not necessarily be representative of fluid management layer material actually used as a component of an absorbent article, which would ordinarily be wound on a roll for storage and shipping, following its manufacture.

The fluid management layer contemplated herein can have a basis weight of up to 75 grams per square meter (gsm); or a basis weight of up to 70 gsm; or a basis weight of about30 gsm to 75 gsm, or more preferably about 45 gsm to 70 gsm, or even more preferably about 50 gsm to 65 gsm, including any values within these ranges and any ranges created thereby.

Some types of absorbent articles may not require a fluid management layer having basis weight as great as set forth above. For example, panty liners, which generally are not placed under the same demand and do not have the same level of absorbent capacity as menstrual pads, may be appropriate for inclusion of a fluid management layer of a lesser basis weight as compared with those set forth above. For example, the fluid management layer for a panty liner may be manufactured to a basis weight of about 20 gsm to 70 gsm, or about 35 gsm to 65 gsm, or even about 40 gsm to 60 gsm, specifically including all values within these ranges and any ranges created thereby. In one specific example, the fluid management layer of the present disclosure can be manufactured to a basis weight of about 45 gsm to 55 gsm. The basis weight of a fluid management layer may be determined by the Basis Weight measurement method set forth below.

The inventors have also found that the processing technique for creating caliper in the fluid management layer can be utilized not only on spunlace materials where the strata are heterogeneous, but also where the strata are homogeneous, e.g., each stratum has the same fiber constitution. Additionally, the inventors have surprisingly found that spunlace materials manufactured with this process, along with appropriate fiber selection, can also provide good resiliency and recovery from compression, with improved fluid handling performance above those spunlace materials that are produced via typical spunlace processes.

As a result of fiber integration, inclusion of an adhesive or binder within the constituent materials of the fluid management layer is not required to add sufficient tensile strength and/or stability. Additionally, the carded nonwoven of the fluid management layers contemplated herein may be manufactured from various suitable fiber types that produce the desired performance characteristics. In examples contemplated herein, the fluid management layer may include a combination of stiffening fibers, absorbent fibers and resilient fibers.

Absorbent fibers may be included to impart the fluid management layer with the ability to absorb discharged fluid. Stiffening fibers may be included, to serve to bond together upon heat treatment of the web, thereby imparting greater stiffness and resiliency to the fluid management layer. Resilient fibers may be included to impart the web with enhanced ability to recover it shape and caliper following application of compressive forces thereto.

To enhance the stabilizing effect of the integration, crimped, carded fibers may be included. One or more of the absorbent fibers, stiffening fibers, and resilient fibers may be crimped prior to integration. For example, where synthetic fibers are utilized, these fibers may be mechanically crimped via passage through the nip between a pair of rollers having intermeshing teeth. Absorbent fibers may be mechanically crimped and/or may be imparted with a chemically induced crimp.

The quantity of absorbent fibers included can impact the ability of the fluid management layer to drawing discharged fluid through and/or from the topsheet. However, when absorbent fibers absorb liquid, they tend to lose some of their structural integrity. The loss of structural integrity can reduce the resiliency of the fluid management layer, and cause or exacerbate bunching and increased leakage. Accordingly, although in principle, a relatively large fraction of absorbent fibers in the fluid management layer may cause it to be comparatively more effective at draining discharged fluid from the topsheet rapidly, this can also lead to other problems or shortcomings with the absorbent article, as mentioned above.

In view of the potential problems associated with having too great of a proportion of absorbent fibers in the fluid management layer, the inventors have found that an appropriate weight fraction of absorbent fibers in the fluid management layer, for balancing the advantages and disadvantages identified above, may be about 10 percent to 60 percent, more preferably about 15 percent to 50 percent, and even more preferably about 20 percent to 40 percent, specifically including any values within these ranges and any ranges created thereby of absorbent fibers. In one particular example, the fluid management layer may include about 20 percent to 30 percent by weight absorbent fibers. The weight fractions of absorbent fibers, resilient fibers, and/or stiffening fibers may be determined via the Material Compositional Analysis method disclosed below.

To mitigate the reduction of structural integrity in the fluid management layer caused by wetting of the absorbent fibers, the fluid management layer may also be constituted of an appropriate weight fraction of resilient fibers, which enhance the ability of the fluid management layer to recover its shape and/or caliper following application of compressive loads that are imposed during use. Accordingly, the fluid management layer may be constituted to include about 15 percent to 70 percent, or about 20 percent to 60 percent, or about 25 percent to 50 percent, by weight, of resilient fibers, specifically reciting all values within these ranges and any ranges created thereby. In one specific example, the fluid management layer may include about 30 percent to 40 percent by weight resilient fibers.

Stiffening fibers may be included to further enhance resiliency of the fluid management layer, and as a result, to the absorbent article. Following fiber blending, accumulation and laydown, stiffening fibers within the precursor fiber accumulation may be bonded to one another via heat treatment of the fluid management layer material. This bonding of the stiffening fibers creates a support matrix which enhances resiliency and stiffness of the fluid management layer. Accordingly, the fluid management layer may be constituted of about 25 percent to 70 percent, or about 30 percent to 60 percent, or even about 40 percent to 55 percent, by weight stiffening fibers, specifically reciting all values within these ranges and any ranges created thereby. In one specific example, the fluid management layer may include about 40 percent to 50 percent by weight stiffening fibers.

Where caliper, resiliency, and a soft cushiony feel are objectives, the weight fraction of stiffening fibers may be greater than or substantially equal to the weight fraction of resilient fibers in the fluid management layer. The weight fraction of absorbent fibers may be less than the weight fractions of resilient fibers and/or stiffening fibers. In general, a higher weight fraction of absorbent fibers is considered to be beneficial in rapid acquisition of discharged fluid from the topsheet; however, where absorbent fibers are disposed proximate to the topsheet, it may be beneficial for the absorbent structure beneath the fluid management layer to draw fluid from the absorbing fibers. Where there is a relatively greater weight fraction of absorbing fibers in the fluid management layer, a relatively a larger absorbent structure therebeneath may be required to drain the absorbent fibers in the fluid management layer, of fluid. This generally imposes greater material costs. Accordingly, an appropriate balance in the weight ratio of absorbent fibers to stiffening fibers may be selected to be about 1:7 to 2:1, or 1:4 to 1.5:1, or even 1:2 to 1:1, specifically reciting all values within these ranges and any ranges created thereby. Similarly, a weight ratio of absorbent fibers to resilient fibers may be selected be about 1:7 to 3:1, or 1:2 to 2:1, or even 1:1.5 to 1:1.

Regardless of whether the fluid management layer is included in an adult incontinence article, menstrual article, liner, or other hygiene article, the ability of the fluid management layer to acquire and draw fluid from the topsheet and to wick the fluid to locations below the topsheet, so that the topsheet does not feel wet to the user/wearer at times following a discharge of fluid. To accomplish this, the inventors have found that the relatively increased caliper of the fluid management layer manufactured as discussed herein can enhance fluid acquisition via the relatively increased void volume within the fluid management layer. Relatively greater caliper at a given basis weight equals relatively greater void volume and higher permeability. Additionally, the relatively greater caliper of the fluid management layer can also increase the level of opacity of the fluid management layer and thereby increase fluid stain masking effects.

Careful selection of the types of fibers constituting each of the strata in the fluid management layer, and selection of the linear densities of the fibers (which correlate with fiber size) can meet the desired objectives of suitably rapid acquisition and low rewet. The fiber types of the individual strata are discussed below.

For purposes herein, reference to a “first” stratum, “second stratum,” etc. below refer to the order of appearance of the strata from top to bottom, beneath the topsheet. The “first” stratum will be at the top of the fluid management layer, closest the topsheet, and so on.

Suitable linear density values of absorbent fibers (expressed in decitex units, or “dtex”) for use in the fluid management layers contemplated herein are as follows. In some examples, the average linear density for absorbent fibers may be selected to be about 1 dtex to 7 dtex, or about 1.4 dtex to 6 dtex, or even about 1.7 dtex to 5 dtex, specifically reciting all values within these ranges and any ranges created thereby. In one specific example, the absorbent fibers included may be selected to have an average linear density of about 0.6 to 2.4 dtex, more preferably about 0.9 to 2.1 dtex, even more preferably about 1.1 to 1.9 dtex, and most preferably about 1.3 to 1.7 dtex. (The average decitex of the absorbent fibers, stiffening fibers, and resilient fibers, if this information is not known or available from the manufacturer or supplier, may be determined via the Fiber Decitex method disclosed herein.)

The absorbent fibers of the fluid management layer may have any suitable cross-section profile shape (where the cross-section lies along a plane that is perpendicular with the greater length dimension of the fiber when it is straight). Some examples of suitable shapes may include trilobal, “H,” “Y,” “X,” “T,” round, or flat ribbon. Further, the absorbing fibers can have cross sections that are solid, hollow or multi-hollow. Other examples of suitable multi-lobed, absorbent fibers for utilization in the fluid management layers described herein are disclosed in U.S. Pat. Nos. 6,333,108; 5,634,914; and 5,458,835. A trilobal fiber shape can improve wicking and improve opacity and stain concealment properties. Suitable trilobal rayon fibers are available from Kelheim Fibres GmbH (Kelheim, Germany) and sold under the trade name GALAXY. While each stratum may include a different shape of absorbing fiber, much like mentioned above, not all carding equipment may be suited to handle such variation between/among strata. In one specific example, the fluid management layer may include absorbent fibers having a round (circular) shape.

The absorbent fibers may include any suitable absorbent material. Some examples of absorbent fibrous materials include cotton, cellulose (e.g., wood) pulp, regenerated cellulose (rayon, viscose, lyocell, etc.) or combinations thereof. In one example, the fluid management layer 30 may include viscose fibers.

The staple length of the absorbent fibers may be selected to be about 20 mm to 100 mm, or 30 mm to 50 mm, or even 35 mm to 45 mm, specifically reciting all values within these ranges and any ranges created thereby. In general, the fiber length of wood pulp is from about 4 to 6 mm and cannot used in conventional carding machines because the pulp fibers are too short. Accordingly, if wood pulp is desired as a fiber in the fluid management layer, additional processes to blend and add pulp to the carded webs may be required. In some examples, pulp may be airlaid between carded webs with the combination being subsequently integrated. As another example, tissue made from pulp may be utilized in combination with the carded webs and the combination may be subsequently integrated.

As noted previously, in addition to absorbent fibers, the fluid management layer of the present disclosure may include stiffening fibers. Stiffening fibers may be included to help impart structural integrity to the fluid management layer. The stiffening fibers can help increase structural integrity of the fluid management layer in a machine direction and/or in a cross-machine direction, which can facilitate web manipulation during processing of the fluid management layer for incorporation into a disposable absorbent article.

Some examples of suitable linear density values for stiffening fibers may be as follows: The stiffening fibers may selected to be about 1.0 dtex to 6 dtex, or more preferably about 1.5 dtex to 5 dtex, or even more preferably about 2.0 dtex to 4 dtex, specifically reciting all values within these ranges and any ranges created thereby. In a particular example, the stiffening fibers may be about 1.8 dtex to 2.6 dtex, or more preferably about 2.2 dtex.

Suitable examples of stiffening fibers may include bi-component fibers comprising polyethylene and polyethylene terephthalate components or polyethylene terephthalate and co-polyethylene terephthalate components. The components of the bi-component fiber may be arranged in a sheath-core-arrangement, a side-by-side arrangement, a concentric sheath-core arrangement, an eccentric sheath-core arrangement, a trilobal arrangement, or other suitable arrangement. In one specific example, the stiffening fibers may include bi-component fibers having polyethylene/polyethylene terephthalate components arranged in a concentric, sheath-core arrangement, wherein the sheath component comprises polyethylene.

While other materials may be useful in a bicomponent fiber having a sheath-core arrangement, the inventors have found that the stiffness of polyethylene terephthalate, included in the core component, is useful for imparting resilience to the structure. At the same time, the polyethylene component of the stiffening fibers, having a relatively lower melting temperature and included in the sheath component can serve to cause the fibers to bond to one another via heat treatment, wherein a plurality of randomly-disposed fiber-to-fiber bonds may be created throughout the structure. This can add tensile strength to the web in both the machine direction (MD) and the cross-machine direction (CD). Additionally, the bonding between lower-melting-point components of stiffening fibers (e.g., polyethylene-PET sheath-core bicomponent fibers) creates a matrix structure that tends to restrict fiber-to-fiber sliding, thereby increasing the resilience of the material.

One of the benefits provided by inclusion of the stiffening fibers is that the integrated nonwoven may be heat treated following the fiber entanglement process. As the weight fraction of stiffening fibers constituting the fluid management layer or a stratum thereof in increased, more fiber-to-fiber bonds/connection points will be created. Too many bonds/connection points may result in a fluid management layer that is excessively stiff for consumer acceptance, and negatively impact user/wearer perception of comfort and/or softness. As such, selection of the weight fraction of stiffening fibers included in the fluid management layer may be deemed important when designing an absorbent article.

In the heat stiffening process, the heating temperature selection may be impacted, in part, by the constituent composition(s) of the stiffening fibers, the design and operating parameters of the heating equipment, and the web processing speed (i.e., duration of exposure to the heated environment). To impart uniform stiffness across the fluid management layer, the heating equipment and operating parameters should be set up to provide uniform heating to the fluid management layer web. Even small variations in temperature can substantially impact the formation of fiber-to-fiber bonds between the stiffening fibers, and resulting tensile strength of the fluid management layer. An example of a suitable heat stiffening process that may be utilized is air-through heating, in which air heated to the selected heating temperature is blown and/or drawn (via vacuum) through the web along a direction that is approximately orthogonal to the larger planes defined by the web.

As noted above, the fluid management layer of the present disclosure may include resilient fibers. Inclusion of resilient fibers can help the fluid management layer maintain its permeability and recovery of shape and dimensions following compression. Any suitable size fiber may be utilized. For example, the resilient fibers can have a linear density (which will correlate with size/diameter), of about 4 dtex to 15 dtex, or about 5 dtex to 12 dtex, or even about 6 dtex to 10 dtex, specifically reciting all values within these ranges and any ranges created thereby. In one specific example, the fluid management layer may include resilient fibers having variable cross sections, e.g., round and hollow spiral, and/or may include resilient fibers having varying decitex. In yet another specific example, the resilient fibers of the present disclosure may have a decitex of about 10. The resilient fibers may be hollow.

The resilient fibers may be composed of/spun from any suitable thermoplastic, such as polypropylene (PP), polyethylene terephthalate (PET), or other suitable thermoplastics known in the art. Other suitable examples of resilient fiber constituents include polyester/co-extruded polyester. Other suitable examples of resilient fibers may include bi-component fibers such as polyethylene/polypropylene, polyethylene/polyethylene terephthalate, polypropylene/polyethylene terephthalate. These bi-component fibers may be configured as a sheath and a core. Utilization and inclusion of bi-component fibers may provide a cost-effective way to increase basis weight of the material while additionally enabling optimization of the pore size distribution. The staple length of the resilient fibers may be selected to be about 20 mm to 100 mm, or about 30 mm to 50 mm, or even about 35 mm 45 mm. The thermoplastic fibers can have any suitable structure or cross-sectional profile shape. For example, the thermoplastic fibers may be round, or may have other shapes, such as spiral, scalloped oval, trilobal, scalloped ribbon, etc. Further, the resilient fibers included may be solid, hollow or multi-hollow. The resilient fibers selected may be solid and round in cross-sectional profile shape.

Optionally, the resilient fibers may be spiral-crimped or flat-crimped. The resilient fibers may have a crimp value between about 4 and 12 crimps per inch (cpi), or between about 4 and 8 cpi, or between about 5 and 7 cpi, or between about 9 and 10 cpi.

Particular non-limiting examples of resilient fibers may be obtained from Wellman International Ltd/Indorama Ventures (Mullagh, Kells Co. Meath, Republic of Ireland) under the trade designations H1311 and T5974. Other examples of suitable resilient fibers for utilization in the carded staple-fiber nonwovens detailed herein are disclosed in U.S. Pat. No. 7,767,598.

Stiffening fibers and resilient fibers should be carefully selected. For example, while the constituent chemistries of the stiffening fibers and the resilient fibers or components thereof may be similar, it may be desired that composition of resilient fibers should be selected such that their melting temperature is higher than that of composition(s) of the stiffening fibers. Otherwise, during heat treatment, bonds may form between resilient fibers and stiffening fibers, resulting in an overly rigid structure.

For weight fractions of absorbent fibers in the fluid management layer above about 30 percent, within the gsm ranges disclosed herein, the resilient fibers and/or stiffening fibers should be carefully selected. For a soft, cushiony fluid management layer with a caliper factor of at least 0.13 or greater as described herein, the resilient and/or stiffening fibers can be selected to counteract the loss of structural integrity of the absorbent fibers when wet. For example, resilient fibers of relatively higher decitex may be useful to mitigate the loss of rigidity exhibited by the absorbent fibers when wetted. In such instances, resilient fibers may be utilized having a linear density of about 5 dtex to 15 dtex, or about 6 dtex to 12 dtex, or even about 7 dtex to 10 dtex.

Alternatively, or in combination, the stiffening fibers may be selected to further enhance structural integrity. For example, the stiffening fibers may include bi-component fibers in a sheath-core configuration where the sheath is co-polyethylene terephthalate (CoPET). However, with such a material change, additional problems may occur. For example, the joining of materials within the fluid management layer might then only be via adhesive or binder as opposed to fusion bonding effected by heat treatment.

In other examples, the materials selections and processes may be configured to effect greater numbers of bonds between stiffening fibers. When the absorbent fibers constitute more than about 30 percent by weight of the fluid management layer, the heat at which the stiffening fibers are bonded may be increased and/or the time of exposure may be increased. This can increase the number of bonds in the stiffening fiber matrix which can mitigate the loss of rigidity of the absorbent fibers when wetted. However, with the increase in the number of bonds comes an increase in stiffness. The increase in stiffness can decrease the perception of softness by the user.

In a similar regard, in addition to or alternatively thereto, the linear density of the stiffening fibers may be increased to mitigate the loss of rigidity of the absorbent fibers, where the absorbent fibers make up about 30 percent by weight or more. In such instances, the linear density of the stiffening fibers may be selected to be about 3 dtex to 6 dtex, or about 4 dtex to 6 dtex.

While it might appear that the solution to “wet collapse” of the fluid management layer is simply to increase the linear density of the stiffening and/or resilient fibers, their selection and proportions should be balanced. Particularly for viscous fluids, the fluid management layers contemplated herein should have some degree of capillarity to help draw fluid from the wearer-facing surface of the article. While the inclusion of relatively higher decitex fibers can have caliper retention benefits, it may reduce capillarity, which reduce the ability of the fluid management layer to draw fluid from the topsheet.

Fluid management layers contemplated herein may be incorporated into a variety of absorbent articles. A non-limiting example of a schematic representation of an absorbent article in the form of a feminine hygiene pad as contemplated herein is shown in FIG. 1A. As reflected, the pad 10 as contemplated herein may include a topsheet 20, a backsheet 50, and an absorbent structure 40 disposed between the topsheet 20 and the backsheet 50. A fluid management layer 30 may be disposed between the topsheet 20 and the absorbent structure 40. The pad has a wearer-facing surface 60 and an opposing outward-facing surface 62. The wearer-facing surface 60 is formed primarily by the topsheet 20 while the outward-facing surface 62 is formed primarily by the backsheet 50. Additional components (not shown) may be included proximate the wearer-facing surface 60 and/or the outward-facing surface 62. For example, if the absorbent article is an incontinence pad, a pair of barrier cuffs which extend generally parallel to a longitudinal axis 100 of the pad 10, and may also form portions of the wearer-facing surface 60. Similarly, one or more deposits fastening adhesive (to be used by the user/wearer to affix the pad within her underwear, at an appropriate location, for use) may be present on the backsheet 50 and form a portion of the outward-facing surface 62 of the absorbent article.

A non-limiting example of a configuration for the fluid management layer 30 is schematically depicted in FIG. 1B. As reflected, the fluid management layer 30 may have opposing end edges 32A and 32B which may extend generally parallel to a lateral axis 200, and side edges 31A and 32B that may extend generally parallel to the longitudinal axis 100. Similarly, the absorbent structure 40 may have opposing end edges 42A and 42B which may extend generally parallel to the lateral axis 200, and side edges 41A and 41B that may extend generally parallel to the longitudinal axis 100.

As reflected in the figures, each of the end edges 32A and 32B of the fluid management layer 30 may be disposed longitudinally outboard of the absorbent structure 40. However, this is not necessarily required. For example, the end edges 32A and/or 32B may be coextensive with the absorbent structure 40 or the end edges 32A and/or 32B may be disposed longitudinally inboard of the end edges 42A and/or 42B of the absorbent structure 40.

Similarly, the side edges 31A and/or 31B may be disposed laterally outboard of the side edges 41A and/or 41B of the absorbent structure 40. Alternatively, the side edges 31A and/or 31B may be laterally coextensive with the side edges 41A and/or 41B of the absorbent structure 40.

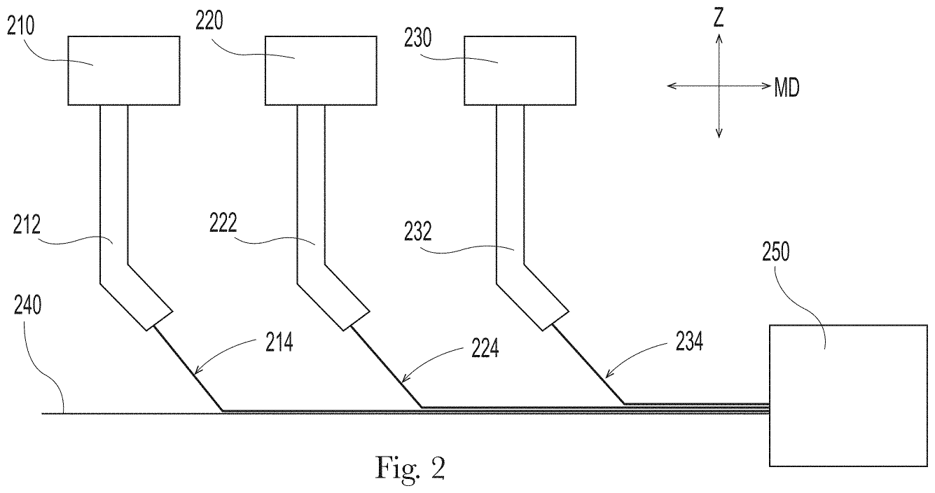

An arrangement of equipment along a manufacturing line configured to perform a process for forming the fluid management layer of the present disclosure is schematically depicted in FIG. 2. As reflected, a plurality of carding machines 210, 220, and 230 may each form a carded web 214, 224, and 234, respectively, which is deposited onto a carrier belt 240 moving along a machine direction MD. Each of the carded webs 214, 224, and 234, may be provided to the carrier belt 240 via a chute 212, 222, 232, respectively. Generally, a carding machine may have a limit on the volume and mass of fiber material it can process and discharge at the desired production rate, and as a consequence, it may impose an upper limit as to the basis weight of the carded fiber web it can produce. Accordingly, for relatively lower basis weight/lower caliper fluid management layers, a single carding machine may be sufficient to lay down an accumulation of carded fibers to reach the desired basis weight at the desired production rate. For a fluid management layer of a greater desired basis weight/greater caliper, two, three or more carding machines may be required, to “stack” carded webs of accumulated fibers to reach the desired basis weight, as suggested in FIG. 2. After the first carded web 214 is deposited on the carrier belt 240, the second carded web 224 is then deposited on/over the first carded web 214 on the carrier belt 240. Similarly, the third carded web 234 (if included) is next deposited on/over the second carded web 224 and the first carded web 214 on the carrier belt 240.

Subsequently, the one or more carded webs 214, 224, and 234 are conveyed to integration equipment 250 which may utilize either needles and/or high-pressure water jets to entangle the fibers of the web(s) and integrate them in the z-direction. Both the carding and integration processes are known in the art.

Fewer, or more, than three carding machines may be utilized in the process. For example, a fluid management layer as contemplated herein may be produced utilizing only two, carding machines. In such example, the first carded web 214 would be deposited on the carrier belt 240. Subsequently, a second carded web 224 would be deposited on/over the first carded web 214. Then, the first carded web 214 and the second carded web 224 would be integrated as described herein.

With the arrangement of equipment schematically depicted in FIG. 2, a variety of configurations of a fluid management layer may be manufactured. However, it is an objective that the fluid management layer have adequate pore volume to allow for rapid acquisition of fluid yet also are able to retain fluid away from the topsheet to reduce the chances of rewetting. With these design objectives in mind, the carded webs, e.g., 214, 224, and/or 234, the fiber compositions of carded webs may be selected to be different from one another. Assuming the first carded web would be closest to the wearer-facing surface of an absorbent article, the fiber selection for the first carded web 214 may be such that there is more pore volume associated with this web. The second carded web 224 may be similarly configured. In contrast, the third carded web 234 may have a fiber composition adapted to draw fluid from the void spaces/pore volumes in the first and second carded webs 214 and 224, and to distribute the fluid to and across an absorbent structure. Alternatively, the first carded web 214, the second carded web 224 and the third carded web 234 may have similar fiber compositions.

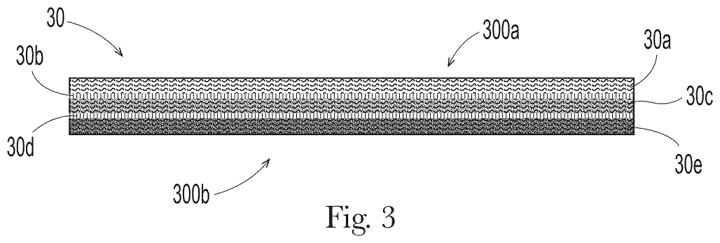

A schematic depiction of non-limiting example of a cross section through a plane along the z-direction, of a fluid management layer as contemplated herein is provided in FIG. 3. As shown, the fluid management layer 30 has a first surface 300a, which is the wearer-facing surface of layer 30, and an opposing second surface 300b, which is the outward-facing surface. Between the first surface 300a and the second surface 300b, the fluid distribution layer 30 may have two or more identifiable strata 30a, 30b, 30c along the z-direction, resulting from the successive laydown of carded webs. The strata may be roughly delineated by fiber integrated interfacial zones 30b, 30d, wherein fibers of one carded web have been integrated with fibers of a superadjacent or subjacent carded web, via a process described above.

Examples of suitable fluid management layers (also known as, for example, “secondary topsheets,” or “acquisition/distribution layers”) are further described in U.S. application Ser. Nos. 16/831,862; 16/831,854; 16/832,270; 16/831,865; 16/831,868; 16/831,870; 16/831,879, and 17/490,193; and U.S. Provisional Apps. Ser. No. 63/086,701. Additional suitable examples are described in U.S. Pat. No 9,504,613; WO 2012/040315, and US 2019/0021917.

### Absorbent Structure

The absorbent structure 40 of the present disclosure may have any suitable shape including but not limited to oval, a stadium, rectangle, an asymmetric shape, peanut, trapezoid, rounded trapezoid, ovoid, and hourglass. In some examples, absorbent structure 40 may have a contoured shape, e.g., one that is narrower in the longitudinally intermediate region than in the end regions. In other examples, the absorbent structure may have a tapered shape that is a wider in one end region of the pad, and tapers to a narrower width in the other end region of the pad. The absorbent structure 40 may have varying stiffnesses in the MD and CD.

The configuration and construction of the absorbent structure 40 may vary (e.g., the absorbent structure 40 may have varying caliper zones, a hydrophilic gradient, a superabsorbent gradient, or lower average density and lower average basis weight acquisition zones). Further, the size and absorbent capacity of the absorbent structure 40 may also be varied to accommodate a variety of wearers. However, the total absorbent capacity of the absorbent structure 40 should be compatible with the design loading and the intended use of the disposable absorbent article or incontinence pad 10.

In some examples, the absorbent structure 40 may include a plurality of layers each having particular features and/or functions. Is some examples, the absorbent structure 40 may include a wrap (not shown) included to envelope enveloping the absorbent constituents of the absorbent structure. The wrap may be formed by one or more nonwoven materials, tissues, films or other materials, or laminates thereof. In one form, the wrap may be formed of only a single material, substrate, laminate, or other material that is wrapped at least partially around itself.

The absorbent structure 40 may include one or more adhesives, for example, to help immobilize the SAP or other absorbent materials within the first and second laminates.

Suitable absorbent structures comprising relatively high amounts of superabsorbent polymer (“SAP”—also known as “absorbent gelling material,” or “AGM”) with various core designs are disclosed in U.S. Pat. No. 5,599,335; EP 1 447 066; WO 95/11652; US 2008/0312622A1; and WO 2012/052172

Additions to the absorbent structure are contemplated. Potential additions to the absorbent structure are described in U.S. Pat. Nos. 4,610,678; 4,673,402; 4,888,231; and 4,834,735. The absorbent structure may further include layers that mimic the dual core system containing an acquisition/distribution core of chemically stiffened fibers positioned over an absorbent storage core as described in U.S. Pat. No. 5,234,423; and in U.S. Pat. No. 5,147,345. These may be deemed useful to the extent they do not negate or conflict with the effects of the below described laminates of the absorbent structure of the present invention.

Some further examples of a suitable absorbent structures 40 that can be used in the absorbent article of the present disclosure are described in US 2018/0098893 and US 2018/0098891.

As noted above, absorbent articles including a fluid management layer contemplated herein may include aa storage layer. Referring back to FIGS. 1A and 1B, a storage layer would generally be positioned at a location corresponding to that in which the absorbent structure 40 is depicted. The storage layer may be constructed as described regarding absorbent structures. The storage layer may contain conventional absorbent materials. In addition to conventional absorbent materials such as creped cellulose wadding, fluffed cellulose fibers, rayon or viscose fibers and comminuted wood pulp fibers (also known as airfelt or fluff pulp), and textile fibers, the storage layer may also include particles or fibers of superabsorbent material that imbibes fluids and forms hydrogels. (Such materials are also known as absorbent gelling materials (AGM).) AGM is typically capable of absorbing a relatively large weight quantity of body fluid per dry weight AGM, retaining it under moderate pressure. Synthetic fibers spun from polymers such as cellulose acetate, polyvinyl fluoride, polyvinylidene chloride, acrylics (such as ORLON), polyvinyl acetate, non-soluble polyvinyl alcohol, polyethylene, polypropylene, polyamides (such as nylon), polyesters, bi-component fibers, tricomponent fibers, mixtures thereof and the like can also be included in the secondary storage layer. The storage layer may also include filler materials, such as PERLITE, diatomaceous earth, VERMICULITE, or other suitable materials, that can serve to reduce changes of rewetting.

The storage layer or fluid storage layer may include absorbent gelling material (AGM) in a uniform distribution throughout, or may include it in a non-uniform distribution. The AGM may be distributed and/or concentrated via deposit thereof into channels or pockets, or may be deposited in patterns including stripes, crisscross patterns, swirls, dots, or any other pattern, either two or three dimensional, that can be imagined. The AGM may be sandwiched between a pair of fibrous cover layers. AGM may be encapsulated, at least in part, by a single fibrous cover layer.

Portions of the storage layer may be formed substantially only of superabsorbent material/AGM, or may be formed of AGM distributed and dispersed in a suitable carrier structure such as a batt or accumulation of cellulose fibers in the form of fluff or stiffened fibers. One non-limiting example of a storage layer may include a first layer formed substantially only of AGM particles or fibers, that are placed or deposited onto a second layer that is formed of a distribution of AGM particles or fibers, within cellulose fibers.

Examples of absorbent structures formed of layers of superabsorbent material/AGM and/or layers of superabsorbent material/AGM dispersed within a batt or other accumulation of cellulose fibers, that may be utilized in the absorbent articles (e.g., sanitary napkins, incontinence products) contemplated herein are disclosed in US 2010/0228209A1. Absorbent structures comprising relatively high amounts of SAP/AGM with various core designs are disclosed in U.S. Pat. No. 5,599,335; EP 1 447 066; WO 95/11652; US. 2008/0312622A1; WO 2012/052172; U.S. Pat. Nos. 8,466,336; and 9,693,910 to Carlucci. These may be used to configure the absorbent structure or storage layer.

Backsheet

The backsheet 50 may be disposed beneath the absorbent structure 40 and be the outwardmost layer of the article, forming the outward-facing surface of the article. The backsheet 50 may be joined to the absorbent structure 40 and/or to the topsheet (about the outer periphery) by any suitable attachment methods known in the art. For example, the backsheet 50 may be secured to the absorbent structure 40 by a uniform continuous layer of adhesive, a patterned layer of adhesive, or an array of separate lines, spirals, or spots of adhesive. Alternatively, the attachment methods may comprise using heat bonds, pressure bonds, ultrasonic bonds, dynamic mechanical bonds, or any other suitable attachment methods or combinations of these attachment methods as are known in the art.

The backsheet 50 may be impervious, or substantially impervious, to liquids (e.g., urine, menstrual fluid) under ordinary conditions of use, and may be manufactured from a thin plastic film, although other flexible liquid impervious materials may also be used. The backsheet 50 may prevent, or at least inhibit, exudates absorbed and contained in the absorbent structure 40 from wetting underwear, outer clothing, bedding, etc. which may come into contact with or proximity to the article 10. However, in some examples the backsheet 50 may be configured so as permit vapor to escape from the absorbent structure 40 (i.e., is “breathable”) while in examples the backsheet 50 may be configured so as to be vapor-impermeable (i.e., non-breathable). Backsheet 50 may include a polymeric film such as a film of polyethylene or polypropylene. A suitable material for the backsheet 50 is a thermoplastic film having a thickness of approximately 0.012 mm (0.5 mil) to 0.051 mm (2.0 mils), for example. Any suitable liquid impermeable backsheet known in the art may be utilized with the present invention.

The backsheet 50 serves as a barrier to prevent migration of fluids absorbed and retained in the absorbent structure 40, to the outward-facing surface of the pad. A preferred material is a soft, smooth, compliant, liquid and vapor pervious material that provides for softness and conformability for comfort, and is low noise producing so that movement does not cause unwanted sound.

Non-limiting examples of materials suitable for forming backsheets are described in U.S. Pat. Nos. 5,885,265; 6,462,251; 6,623,464; and 6,664,439. Examples of suitable dual- or multi-layer breathable backsheets include those described in U.S. Pat. Nos. 3,881,489; 4,341,216; 4,713,068; 4,818,600; EP 203 821; EP 710 471; EP 710 472; and EP 793 952. Additional examples of suitable single layer breathable backsheets for include those described in GB A 2184 389; GB A 2184 390; GB A 2184 391; U.S. Pat. Nos. 4,591,523; 3,989,867; 3,156,242; and WO 97/24097.

The backsheet may be a nonwoven web having a basis weight of about 20 gsm to 50 gsm. In one example, the backsheet may be a hydrophobic 23 gsm spunbond nonwoven web of 4 denier polypropylene fibers, available from Fiberweb Neuberger, under the trade designation F102301001. The backsheet may be coated with a non-soluble, liquid swellable material as described in U.S. Pat. No. 6,436,508.

The backsheet has an outward-facing side and an opposing wearer-facing side. The outward-facing side of the backsheet may include a non-adhesive area and an adhesive area. The adhesive area may be provided by any conventional means, for the purpose of enabling the user/wearer to affix the pad to the wearer-facing surface of her underwear at a location suitable for use. Pressure-sensitive adhesives have been found to work well for this purpose.

Experimentation

For purposes of experimentation and discovery, the inventors conducted manufacture and testing of quantities of prototype samples of absorbent articles in the form of feminine hygiene pads including various configurations of topsheets and fluid management layers as contemplated herein. The prototype samples had the following features.

### Topsheets

Topsheets for all prototype samples had a basis weight of about 24 gsm.

Topsheets for all prototype samples were cut from a nonwoven web of carded staple length fibers having an average staple length of 38 mm and an average denier of 2. The fibers were bicomponent, with a concentric sheath-core configuration, in which the core component was PET and the sheath component was LDPE; the weight ratio of the components was 1:1. The nonwoven web included a plurality of randomly-distributed fiber-to-fiber bonds that had been created via air-through heat bonding of the carded fibers.

The topsheets were affixed to the subjacently-disposed fluid management layers via application of a pressure sensitive adhesive applied in a discontinuous series of narrow spiral paths generally oriented in the longitudinal direction. The fluid management layers were affixed to the subjacently-disposed absorbent structures in a similar manner.

Further details varied among samples as specified below:

Sample #1: The topsheet nonwoven was formed of a blend of hydrophilic and hydrophobic fibers, in a ratio of 60:40, weight of hydrophilic fibers to weight of hydrophobic fibers.

Sample #2: The topsheet nonwoven was formed of a blend of hydrophilic and hydrophobic fibers, in a ratio of 60:40, weight of hydrophilic fibers to weight of hydrophobic fibers. The topsheet bore an application of anti-stick agent consisting of about 1 percent by weight PPG-15 stearyl ether and about 99 percent by weight caprylic/capric triglyceride, the anti-stick agent having been sprayed on to the wearer-facing surface at an application level of about 2 gsm.

Sample #3: The topsheet nonwoven was formed of a blend of hydrophilic and hydrophobic fibers, in a ratio of 60:40, weight of hydrophilic fibers to weight of hydrophobic fibers. The nonwoven had a regular pattern of apertures therethrough, the apertures having an average size of 0.55 mm² and constituting an open area of about 3 percent.

Sample #4: The topsheet nonwoven was formed of a blend of hydrophilic and hydrophobic fibers, in a ratio of 60:40, weight of hydrophilic fibers to weight of hydrophobic fibers. The nonwoven had a regular pattern of apertures therethrough, the apertures having an average size of 0.60 mm² and constituting an open area of about 12 percent.

Sample #5: The topsheet nonwoven was formed of a blend of hydrophilic and hydrophobic fibers, in a ratio of 60:40, weight of hydrophilic fibers to weight of hydrophobic fibers. The nonwoven had a regular pattern of apertures therethrough, the apertures having an average size of 0.60 mm² and constituting an open area of about 12 percent. The topsheet bore an application of anti-stick agent consisting of about 1 percent by weight PPG-15 stearyl ether and about 99 percent by weight caprylic/capric triglyceride, the anti-stick agent having been sprayed on to the wearer-facing surface at an application level of about 2 gsm.

Sample #6: The topsheet nonwoven was formed of a blend of hydrophilic and hydrophobic fibers, in a ratio of 60:40, weight of hydrophilic fibers to weight of hydrophobic fibers. The nonwoven had a regular pattern of apertures therethrough, the apertures having an average size of 2.3 mm² and constituting an open area of about 19 percent.

Sample #7: The topsheet nonwoven was formed of entirely hydrophobic fibers, and had no apertures and no application of anti-stick agent.

Sample #8: The topsheet nonwoven was formed of entirely hydrophobic fibers. The nonwoven had a regular pattern of apertures therethrough, the apertures having an average size of 0.55 mm² and constituting an open area of about 3 percent.

Sample #9: The topsheet nonwoven was formed of entirely hydrophobic fibers. The nonwoven had a regular pattern of apertures therethrough, the apertures having an average size of 0.60 mm² and constituting an open area of about 12 percent.

Sample #10: The topsheet nonwoven was formed of entirely hydrophobic fibers. The nonwoven had a regular pattern of apertures therethrough, the apertures having an average size of 2.30 mm² and constituting an open area of about 19 percent.

### Fluid Management Layer

All prototype samples included a fluid management layer of a substantially common structure and composition, disposed subjacent the topsheet. The common fluid management layer had a basis weight of about 65 gsm. It was constituted of about 20 percent by weight, 1.3 dtex viscose fibers; about 30 percent by weight, 10 dtex hollow spiral polyethylene terephthalate fibers; and about 50 percent by weight, 2.2 dtex bi-component fibers having a concentric sheath-core configuration, wherein the core component was PET and the sheath component was PE, in a weight ratio of PET:PE of about 1:1. These bicomponent fibers had an average decitex of about 2.2. The fluid management layer had two strata each having the same homogeneous blend of fibers, was lightly hydroentangled, and was air-through heat bonded.

### Absorbent Structure

All prototype samples included an absorbent structure of a substantially common structure and composition, disposed subjacent the fluid management layer. The common absorbent structure was an airlaid blend of pulp fibers, absorbent gelling material, and bicomponent fibers, having a basis weight of 182 gsm, available pre-manufactured in festooned web form from Glatfelter Corporation (Charlotte, N.C., USA). It is not believed that the structure and composition of the absorbent structure had any significant role in the differences in performance measured among the various prototype samples.

### Backsheet

All prototype samples included a common backsheet disposed beneath the absorbent structure, formed from a sheet of extruded polyethylene film.

### Test Results and Data

Quantities of all 10 prototype samples were subjected to measurement and testing using the Acquisition Time and Rewet Measurement Method set forth below.

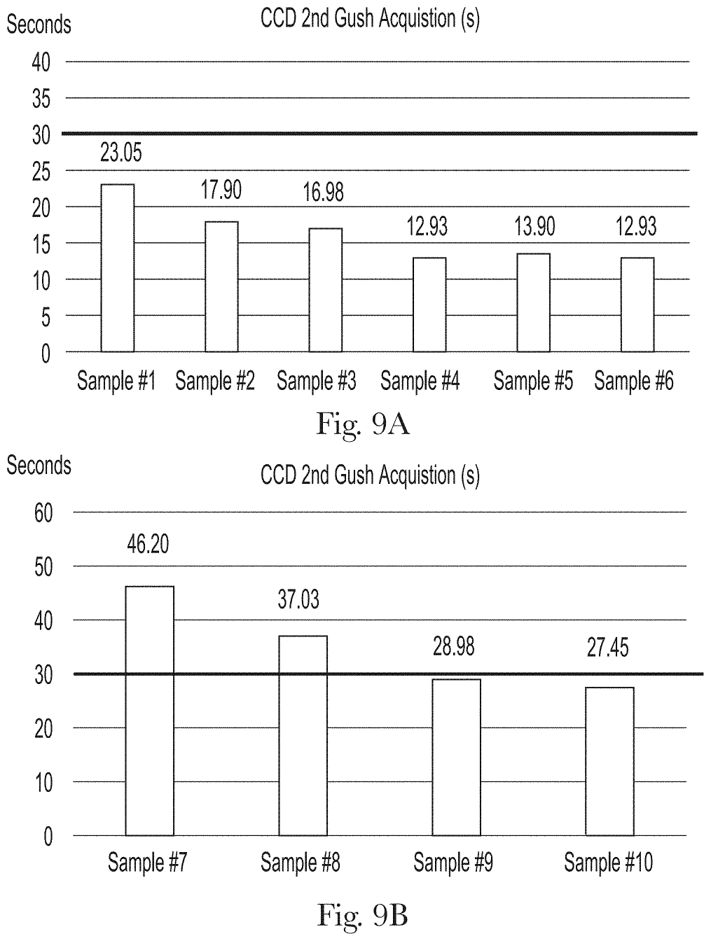

FIGS. 9A and 9B report the second acquisition time (ACQ-2) measured for each of the ten samples. For purposes herein, it is believed that the second acquisition time (ACQ-2) is most relevant to the actual user experience because it reflects fluid acquisition for a feminine hygiene pad that has been in use/worn for some duration and has absorbed some fluid, but is then is exposed to a relatively larger discharge of fluid as might occur with a sudden change of the user's/wearer's body position following a period of inactivity or relatively low activity. The heavy horizontal line drawn at the 30-second mark in the charts represents what the inventors believe to be the highest acquisition time that is acceptable to the relevant consumers/users.

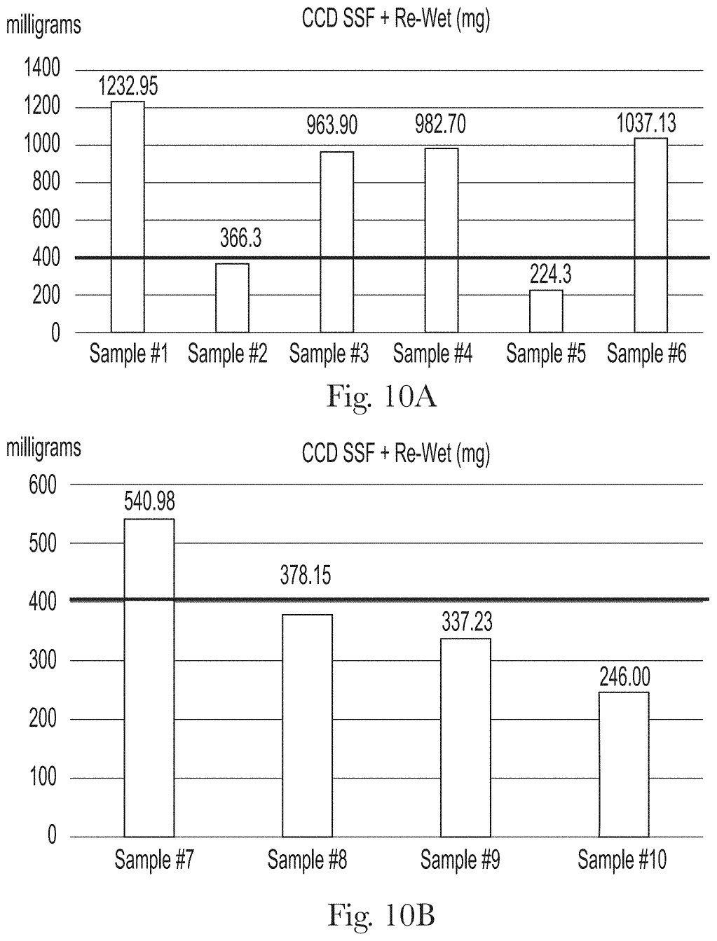

FIGS. 10A and 10B report the sum of surface free fluid (SFF) and rewet measured for each of the ten samples. For purposes herein, it is believed that this sum is most relevant to the actual user experience because it reflects the extent to which a pad which has absorbed a substantial quantity of fluid, will permit the fluid to return to the surface of the topsheet under moderate pressure, which reflects cause of an unsatisfactory wet feeling for the user/wearer. The heavy horizontal line drawn at the 400-milligram mark in the charts represents what the inventors believe to be the highest SFF+rewet that is acceptable to the relevant consumers/users.

From the charts in FIGS. 9A, 9B, it can be seen that prototype samples 1-6, 9 and 10 exhibited second acquisition times that are deemed acceptable, while prototype samples 7 and 8 did not.

From the charts in FIGS. 10A and 10B, it can be seen that prototype samples 2, 5 and 8-10 exhibited SFF+rewet values that are deemed acceptable, while prototype samples 1, 3, 4, 6 and 7 did not.

From this data it might be concluded that prototype samples 2, 5, 9 and 10 were the most successful, according to the assessment herein.

Test and Measurement Methods

### Caliper

The caliper, or thickness, of a test specimen is measured as the distance between a reference platform on which the specimen rests and a pressure foot that exerts a specified amount of pressure onto the specimen over a specified amount of time. All measurements are performed in a laboratory maintained at 23° C.±2 C.° and 50%±2% relative humidity and test specimens are conditioned in this environment for at least 2 hours prior to testing.

Caliper is measured with a manually-operated micrometer equipped with a pressure foot capable of exerting a steady pressure of 0.50 kPa±0.01 kPa onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm, however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen by removing it from an absorbent article, if necessary. When excising the test specimen from an absorbent article, use care to not impart any contamination or distortion to the test specimen layer during the process. The test specimen is obtained from an area free of folds or wrinkles, and it must be larger than the pressure foot.

To measure caliper, first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the caliper of the test specimen to the nearest 0.001 mm. In like fashion, repeat for a total of ten replicate test specimens. Calculate the arithmetic mean for all caliper measurements and report as Caliper to the nearest 0.001 mm.

### Caliper Factor

The caliper factor, as mentioned previously is the caliper per 10 gsm of basis weight of the sample. So, the equation is caliper/(basis weight/10).

### Basis Weight

The basis weight of a sample of sheet or web material is the mass (in grams) per unit area (in square meters) of a single layer of the material. If it is not otherwise known or available, basis weight may be measured using EDANA compendial method NWSP 130.1. The mass of the test sample is cut to a known area, and the mass of the sample is determined using an analytical balance accurate to 0.0001 grams. All measurements are performed in a laboratory maintained at 23° C.±2 C.° and 50%±2% relative humidity and test samples are conditioned in this environment for at least 2 hours prior to testing.

Measurements are made on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen must be as large as possible so that any inherent material variability is accounted for.

Measure the dimensions of the single layer test specimen using a calibrated steel metal ruler traceable to NIST, or equivalent. Calculate the Area of the test specimen and record to the nearest 0.0001 square meter. Use an analytical balance to obtain the Mass of the test specimen and record to the nearest 0.0001 gram. Calculate Basis Weight by dividing Mass (in grams) by Area (in square meters) and record to the nearest 0.01 grams per square meter (gsm). In like fashion, repeat for a total of ten replicate test specimens. Calculate the arithmetic mean for Basis Weight and report to the nearest 0.01 grams/square meter.

### Material Compositional Analysis

If the information is not otherwise available, the quantitative chemical composition of a test specimen comprising a mixture of fiber types is determined using ISO 1833-1. All measurements are performed in a laboratory maintained at 23° C.±2 C.° and 50%±2% relative humidity.

Analysis is performed on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained and tested as per ISO 1833-1 to quantitatively determine its chemical composition.

### Average Fiber Decitex (dtex) or Denier

Textile webs (e.g., woven, nonwoven, airlaid) are comprised of individual fibers of material. Fibers are characterized in one respect, by their linear mass density, reported in units of denier, or units of decitex. The decitex value is the mass in grams of a fiber present in 10,000 meters of that fiber. The denier value is the mass in grams of a fiber present in 9,000 meters of that fiber. The average decitex or denier value of the fibers within a web of material is often reported by manufacturers as part of a specification. If the average decitex or denier value of the fiber is not otherwise known or available, it can be calculated by measuring the cross-sectional area of the fiber via a suitable microscopy technique such as scanning electron microscopy (SEM), determining the composition of the fiber with suitable techniques such as FT-IR (Fourier Transform Infrared) spectroscopy and/or DSC (Dynamic Scanning Calorimetry), and then using a literature value for density of the composition to calculate the mass in grams of the fiber present in 10,000 meters of the fiber (for decitex), or in 9,000 meters of the fiber (for denier).

All testing is performed in a room maintained at a temperature of 23° C.±2.0° C. and a relative humidity of 50%±2% and samples are conditioned under the same environmental conditions for at least 2 hours prior to testing.

If necessary, a representative sample of web material of interest can be excised from an absorbent article. In this case, the web material is removed so as not to stretch, distort, or contaminate the sample.

SEM images are obtained and analyzed as follows to determine the cross-sectional area of a fiber. To analyze the cross section of a sample of web material, a test specimen is prepared as follows. Cut a specimen from the web that is approximately 1.5 cm (height) by 2.5 cm (length) and free from folds or wrinkles. Submerge the specimen in liquid nitrogen and fracture an edge along the specimen's length with a razor blade (VWR Single Edge Industrial Razor blade No. 9, surgical carbon steel). Sputter coat the specimen with gold and then adhere it to an SEM mount using double-sided conductive tape (Cu, 3M available from electron microscopy sciences). The specimen is oriented such that the cross section is as perpendicular as possible to the detector to minimize any oblique distortion in the measured cross sections. An SEM image is obtained at a resolution sufficient to clearly elucidate the cross sections of the fibers present in the specimen. Fiber cross sections may vary in shape, and some fibers may consist of a plurality of individual filaments. Regardless, the area of each of the fiber cross sections is determined (for example, using diameters for round fibers, major and minor axes for elliptical fibers, and image analysis for more complicated shapes). If fiber cross sections indicate inhomogeneous cross-sectional composition, the area of each recognizable component is recorded and dtex contributions are calculated for each component and subsequently summed. For example, if the fiber is bi-component, the cross-sectional area is measured separately for the core and sheath, and dtex contribution from core and sheath are each calculated and summed. If the fiber is hollow, the cross-sectional area excludes the inner portion of the fiber comprised of air, which does not appreciably contribute to fiber dtex. Altogether, at least 100 such measurements of cross-sectional area are made for each fiber type present in the specimen, and the arithmetic mean of the cross-sectional area aₖ for each are recorded in units of micrometers squared (μm²) to the nearest 0.1 μm².

Fiber composition is determined using common characterization techniques such as FTIR spectroscopy. For more complicated fiber compositions (such as polypropylene core/polyethylene sheath bi-component fibers), a combination of common techniques (e.g. FTIR spectroscopy and DSC) may be required to fully characterize the fiber composition. Repeat this process for each fiber type present in the web material.

The average decitex dk value for each fiber type in the web material is calculated as follows:

dₖ=10 000 m×aₖ×ρₖ×10⁻⁶

where dₖ is in units of grams (per calculated 10,000 meter length), aₖ is in units of μm², and ρₖ is in units of grams per cubic centimeter (g/cm³). Average decitex is reported to the nearest 0.1 g (per calculated 10,000 meter length) along with the fiber type (e.g. PP, PET, cellulose, PP/PET bico). The average denier value for each fiber type in the web material is its decitex dₖ value×0.9.

### Apertures Percent Open Area Measurement Method

Percent open area is measured on images, of an apertured topsheet test specimen, acquired using a flatbed scanner. The scanner is capable of scanning in reflectance mode at a resolution of 2400 dpi and 8 bit grayscale. A suitable scanner is an Epson Perfection V750 Pro from Epson America Inc. (Long Beach, Calif., USA) or one having substantially similar functionality. The scanner is interfaced with a computer running an image analysis program. A suitable program is ImageJ v. 1.47 (National Institute of Health, USA), or one having substantially similar functionality. The specimen images are distance calibrated against an acquired image of a ruler certified by NIST. To enable maximum contrast, the specimen is backed with an opaque, background sheet of uniformly black color, prior to acquiring the image. All measurement is performed in a conditioned room maintained at about 23±2° C. and about 50±2% relative humidity.

The measurement specimens are prepared as follows.

Obtain the required number of samples of the absorbent article of interest. To obtain a measurement specimen, tape the sample absorbent article about its periphery (i.e., do not tape over regions underlaid by the fluid management layer), wearer-facing side up, in a flat configuration, to a horizontal flat work surface. Any elastic materials included (e.g., in leg cuffs), if present, may be cut to facilitate laying the article out flat. The outer boundary of the region of the apertured topsheet overlying the fluid acquisition layer of the article is identified and marked. Now cut through the topsheet and any adhered underlying layers, about and through this marked outer boundary with a new razor blade or other comparable new, sharp, cutting implement. From this cut out portion, the test specimen of the apertured topsheet is then carefully separated and removed from the underlying layer(s) such that its longitudinal and lateral dimensions are not changed, to avoid distortion of the apertures. If the topsheet is adhered via an adhesive to an underlying layer, before attempting separation apply any solvent suitable for dissolving the adhesive and allowing easy separation of the topsheet from underlying layer(s) without dissolving the polymer material(s) of fibers constituting the topsheet nonwoven web material. (In many examples, tetrahydrofuran (THF) can be a suitable solvent for this purpose. It is not a concern if the solvent dissolves applied surface finish coatings on the fibers, as long as it does not dissolve the polymer(s) constituting the fibers themselves.) Once the cut-out portion of the topsheet constituting the measurement specimen is removed, identify the wearer-facing side thereof. Five replicate measurement specimens obtained from five samples of the absorbent articles of interest, are prepared for measurement. The specimens are conditioned at about 23° C.±2 C.° and about 50%±2% relative humidity for 2 hours prior to imaging.

Images are obtained as follows.

The ruler is placed on the scanner bed such that it is oriented parallel to the sides of the scanner glass. An image of the ruler (the calibration image) is acquired in reflectance mode at a resolution of 2400 dpi (approximately 94 pixels per mm) and in 8-bit grayscale. The calibration image is saved as an uncompressed TIFF format file. After obtaining the calibration image, the ruler is removed from the scanner glass and all specimens are scanned under the following scanning conditions.

A measurement specimen is placed onto the center of the scanner bed, lying flat, with the body-facing surface of the specimen facing the scanner's glass surface. The corners and edges of the specimen are secured such that its original longitudinal and lateral dimensions, as on the article prior to removal, are retained. The specimen is oriented such that the long axis and short axis thereof are aligned parallel with and perpendicular to the sides of the scanner's glass surface, respectively. The black background is placed on top of the specimen, the scanner lid is closed, and a scanned image of the entire specimen is acquired with the same settings as used for the calibration image. The specimen image is saved as an uncompressed TIFF format file. The remaining four replicate specimens are scanned and saved in like manner.

The specimen image is analyzed as follows. Open the calibration image file in the image analysis program, and calibrate the image resolution using the imaged ruler to determine the number of pixels per millimeter. Now open the specimen image in the image analysis program, and set the distance scale using the image resolution determined from the calibration image. Now identify a rectangular section (region of interest, or “ROI”) longitudinally and laterally centered on the specimen, having a longitudinal dimension along the longitudinal axis of 60.0 mm and a lateral dimension of 30.0 mm, and visually inspect the images of the apertures present within the ROI. Now using the software tools, manually outline each of the apertures within the ROI (and any partial portions thereof at the edges of the ROI). The appropriate outlines will be drawn along visually discernible inside edges of the concentrations of displaced fibers 503 about the perimeters of the apertures. Stray individual fibers that may have escaped the main structure and/or the concentrations of displaced fibers about the perimeter, and cross into or through the main open area of the aperture (by way of illustrative example, stray individual fibers 504 shown in FIG. 5) are not considered subtractive from the aperture area for purposes herein.) Then use the software to measure the area within each discrete aperture outline (whole and partial) within the ROI and record each to the nearest 0.01 mm², and calculate the sum total thereof. The area of each discrete aperture is defined as the x-y surface area within the visually discernable outline of the open region, created by mechanical penetration of the web and x-y direction displacement of fibers in an aperturing process, that creates the apertures through the web. (For example, refer to FIG. 5 where discrete aperture area 501 and visually discernable boundary 502 are depicted. The dark area of the depicted aperture is an image of black construction paper used as a backing to the specimen of which this particular image was made.) The sum of the areas of all of the apertures within the ROI is recorded as Aperture Area to the nearest 0.01 mm². Now divide the Aperture Area by the ROI Area (1,800 mm²), then multiply by 100 and record as Open Area to the nearest 0.1%.

In like manner, repeat the entire procedure for the remaining four replicate specimen images. Calculate the arithmetic mean of Open Area across all five replicate specimens and report as Average Open Area to the nearest 0.1%.

### Acquisition Time and Rewet Measurement Method

This method describes how to measure gush acquisition time, interfacial free fluid amount as well as low and high pressure rewet values for an absorbent article loaded with new Artificial Menstrual Fluid (nAMF), prepared as described herein. A pretreatment step is followed by three introductions of known volumes of nAMF to the absorbent article. The time required for the absorbent article to acquire each of the doses of nAMF is measured using a strikethrough plate and an electronic circuit interval timer. After each liquid dose, Interfacial Free Fluid (IFF) is measured gravimetrically as fluid is transferred from the bottom surface of the strikethrough plate to filter paper. Subsequently, low and high pressure rewet are measured after the last liquid dose. Surface Free Fluid (SFF) is the amount of fluid that remains in the topsheet of the absorbent article. SFF is measured by performing low pressure (0.1 psi) rewet. Immediately after measuring SFF, a higher pressure (0.5 psi) rewet is performed to determine the overall rewet of the absorbent article. All testing is performed in a room maintained at 23° C.±2 C.° and 50%±2% relative humidity.

### Equipment and Supplies

### Strikethrough Plate Configuration

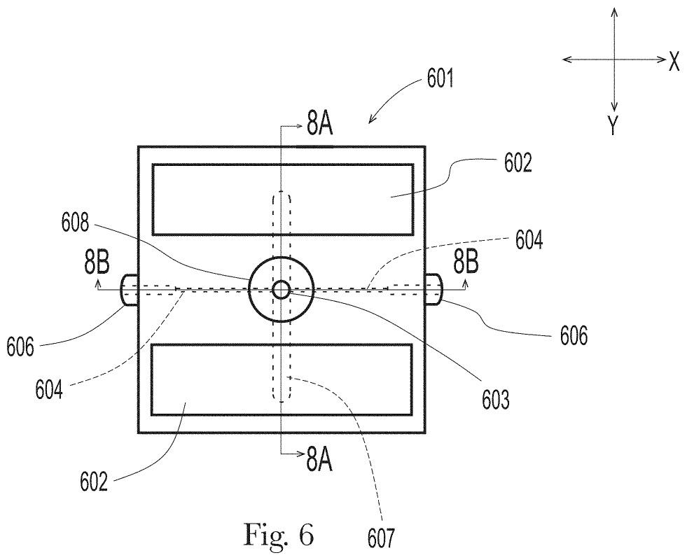

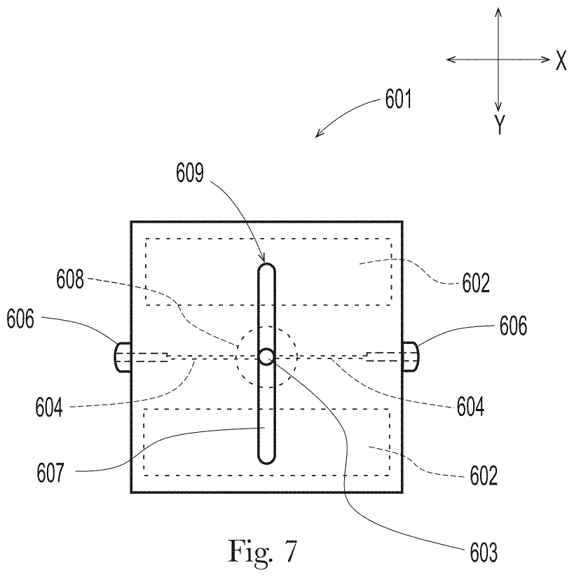

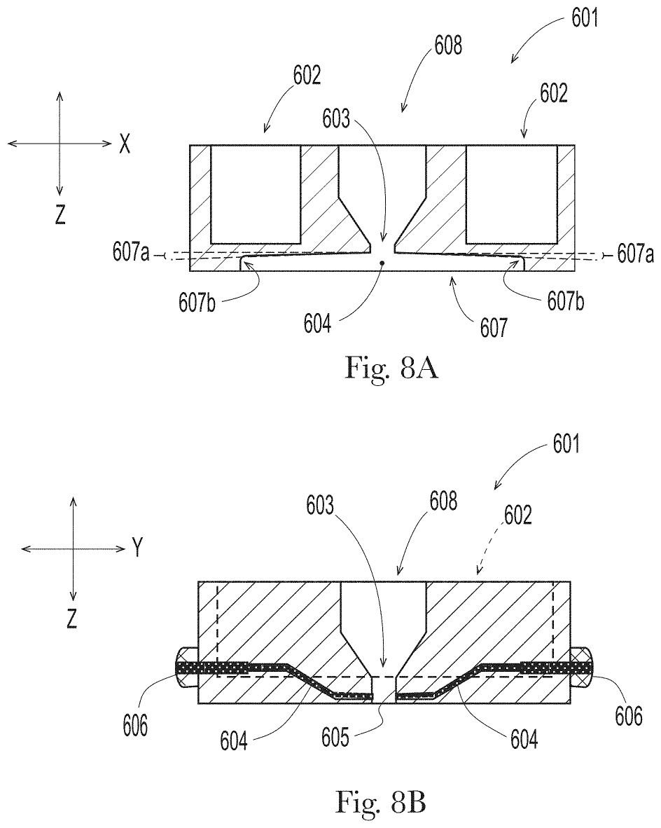

Referring to FIGS. 6, 7, 8A and 8B, the strikethrough plate 601 is made of transparent Plexiglas or equivalent, with an overall dimension of 10.2 cm long (y-direction) by 10.2 cm wide (x-direction) by 3.1 cm tall. (All position and spatial references herein assume an orientation of the strikethrough plate as it would have when resting on a horizontal surface, bottom side down. All references to x-, y- and z-directions in this measurement method description are solely with respect to references to the x-, y- and z-direction arrow indicators appearing in FIGS. 6, 7, 8A and 8B and do not necessarily apply to such references appearing elsewhere in the present specification.) A central, test fluid well 608 with a circular opening and cylindrical wall 25 mm in diameter opens at the top surface of the plate, and extends vertically downwardly (z-direction) from the top surface of the plate to a depth of 15 mm, and then turns radially inwardly to define a conical wall extending vertically downwardly from the top surface by an additional 7.5 mm, while tapering uniformly down to a diameter matching that of the test fluid port 603. The test fluid port 603 is concentric/coaxial with the test fluid well 608 and has a cylindrical wall with a diameter of 6.6 mm, extending further vertically downwardly from the top surface by 5 mm, to a longitudinal fluid channel 607. The longitudinal fluid channel 607 is machined or otherwise formed in the bottom of the plate. The longitudinal fluid channel 607 has a depth from the bottommost surface of the plate defined by vertical side walls that extend upwardly (z-direction) 3.5 mm at the midpoint of the channel (at the test fluid port 603), then slant downwardly at an angle 607a of 0.72° towards each longitudinal end of the channel. The longitudinal fluid channel is open at the bottom surface of the plate, so as to allow fluid to be introduced onto an underlying test specimen, and permitted to flow along the x-y area bounded by the fluid channel 607. The fluid channel 607 is centered under the test fluid port 603 and extends with its length in the in the y-direction that is perpendicular to that of the x-direction paths of the electrodes 604 through the plate. The longitudinal fluid channel 607 has an x-direction width of 5 mm and a y-direction length of 80 mm, upper corners that are rounded with a radius 607b of 1.0 mm about the entire perimeter of the channel. The walls at the opposite distal ends of the longitudinal fluid channel 607 have a cylindrical radius 609 in an x-y plane of 2.5 mm.

Two rectangular cavities 602 (80.5 mm long (x-direction) by 24.5 mm wide (y-direction) by 25 mm deep (z-direction) are symmetrically arranged outboard of the fluid port 603, and centered about a y-direction axis of the plate. These may be loaded with lead shot (or other weighting material) to an extent needed to adjust the total mass of the plate plus the weighting material to provide a pressure of 0.10 psi (7.0 g/cm²) over the total x-y area defined the plate, which is deemed for purposes herein to be 10.2 cm×10.2 cm=104.04 cm², without subtraction of the area defined by the longitudinal fluid channel 607. Electrodes 604 are embedded in the plate 601, each providing an electrical connection between one of the two exterior banana jacks 606 to a position opposite the other, on the inside wall 605 of the longitudinal fluid channel 607. The lowermost portions of the electrodes 604 where their ends are exposed in port 603 is 1.57 mm from the bottommost surface of the plate 601. A circuit interval timer is connected to the jacks 606, so as to monitor the impedance or resistance between the two electrodes 604, and measure the time from introduction of the nAMF into port 603 (establishing an electrical connection between the electrodes and/or substantially decreasing the impedance or resistance therebetween) until the nAMF drains from the port 603 and channel 607 into the test specimen, to a level below the electrodes (breaking the electrical connection between the electrodes and/or substantially increasing the impedance or resistance therebetween). The circuit interval timer has a resolution of 0.01 sec.

### Pretreatment Plate

A pretreatment plate (not shown) is used in combination with a pretreatment weight (not shown) to apply droplets of nAMF to the surface of the test specimen to prime the surface of the specimen prior to the introduction of the full fluid doses specified below. The pretreatment plate is rectangular, made of transparent Plexiglass or equivalent, 14 inch (35.6 cm) long by 8 inch (20.3 cm) wide with a thickness/caliper of about 0.25 inch (6.4 mm). The pretreatment plate is marked with five circular markers, each 5 mm in diameter, placed 1 cm apart (center to center) and centered along the longitudinal axis of the plate. The central marker of the five is centered at the lateral midpoint of the longitudinal axis of the plate. These markers indicate the placement of the nAMF droplets. The markers are located on the underside of the pretreatment plate and can be milled out or simply drawn on with a permanent marker, or equivalent, in any manner such that they are visible through the top surface of the pretreatment plate.

### Pretreatment Weight

The pretreatment weight (not shown) is 10.2 cm×10.2 cm in x- and y-dimensions and consists of a flat, smooth rigid material (e.g., stainless steel). The pretreatment weight has a total mass of 726 g±0.5 g to result in a pressure of 0.10 psi (7.0 g/cm²) across the bottom 104.04 cm² surface area of the pretreatment weight.

### IFF Rubber Pad

When measuring the interfacial fluid amounts, a rubber pad (“IFF rubber pad”) (not shown) with a flat surface is used. The IFF rubber pad is made from high strength neoprene rubber with 40A durometer and a thickness/caliper of ⅛ inch (available from W. W. Grainger, Inc, item #1DUV4, or equivalent) and cut to dimensions of 6 inches (15.2 cm) by 6 inches (15.2 cm).

### Rewet Weight Assembly

For the overall rewet portion of the test, a padded weight assembly (“rewet weight assembly”) (not shown) configured to apply 0.5 psi (35.1 g/cm²) over its 10.2 cm by 10.2 cm (104.04 cm²) surface area is required. The rewet weight assembly is assembled as follows.

Lay a piece of polyethylene film (about 25 microns thick, and about 22.5 cm square in the x-y directions, any convenient source) out flat on a horizontal work surface. A piece of polyurethane foam (25 mm thick, density of 1.0 lb/ft³, IDL 24 psi, available from Concord-Renn Co., Cincinnati, Ohio, or equivalent) is cut to 10.2 cm by 10.2 cm and then laid in a centered position on top of the film. A piece of transparent Plexiglas (10.2 cm by 10.2 cm and about 6.4 mm thick) is then stacked on top of the polyurethane foam. Next, the polyethylene film is gently pulled taut under the polyurethane foam, and portions of the polyethylene film extending outwardly from under the polyurethane foam are wrapped up and over the polyurethane foam and Plexiglas plate, and secured thereabout with transparent tape. A metal weight of suitable mass is selected and stacked on top of, and fastened to, the Plexiglass plate, such that the total mass of the assembly (rewet weight assembly) is 3.6 kg±0.1kg.

### Filter Paper

For the IFF, SFF and overall rewet steps, various numbers of layers of filter paper (not shown) are required. The filter paper to be used is to be conditioned at 23° C.±2 C.° and 50%±2% relative humidity for at least 2 hours prior to testing. A suitable filter paper has a basis weight of about 88 gsm, a thickness of about 249 microns with an absorption rate of about 5 seconds, and is available from Ahlstrom-Munksjo (Mt. Holly Springs, Pa.) as grade 632, or equivalent. Each sheet of the filter paper is square, with dimensions of 5 inches by 5 inches (12.7 cm by 12.7 cm).

### Procedure 1) Test samples (examples of absorbent articles of interest) are conditioned at 23° C.±2 C.° and 50%±2% relative humidity for at least 2 hours prior to testing.

2) Test samples are removed from their outer packaging and the wrappers are opened to unfold the product, if applicable, using care not to press down or pull on the products while handling. No attempt is made to smooth out wrinkles. Using scissors, cut any adhesive-covering release paper connecting the wings, if present, and lay the sample on a horizontal work surface with the wearer-facing surface facing up (i.e., outward-facing side down).

3) For each sample, determine the dose location as follows. The dose location is the intersection of the midpoints of the longitudinal and lateral axes of the fluid management layer. Once the dose location is identified, mark it with a small dot using a black, fine-tip, permanent marker.

4) For each test of a sample, the test sample is pretreated with nAMF as follows.

a) Place the pretreatment plate onto a horizontal work surface such that the side with the circular markers is facing down.

b) Using a single channel, fixed volume pipettor, dispense 50 μL of nAMF onto the topside of the pretreatment plate at each of the five locations overlying each of the five circular markers.

c) Position the test sample above the pretreatment plate with the wearer-facing surface of the sample down, facing the pretreatment plate, such that the longitudinal axes of the sample and of the pretreatment plate are aligned, and the pre-marked dose location on the test sample is centered over the center droplet of nAMF on the pretreatment plate.

d) After it is properly positioned over the pretreatment plate, bring the test sample down into contact with the pretreatment plate, and then promptly place the pretreatment weight over/onto the outward-facing (upward-facing) side of the test sample, centering it over the dose location/central droplet of nAMF on the pretreatment plate, and immediately start a stopwatch-type timer set to alarm after 40 seconds. After 40 seconds have elapsed, remove the pretreatment weight from the test sample and remove the test sample from the pretreatment plate. Flip the test sample over so that the wearer-facing side is facing up, place it onto a horizontal work surface, and promptly proceed with the steps that follow.

5) The first acquisition time (ACQ-1) is measured as follows.

a) Connect the electronic circuit interval timer to the strikethrough plate 601 and zero the timer.

b) Position the strikethrough plate 601 above the wearer-facing surface of the test sample such that the long (y-direction) axis of the longitudinal fluid channel 607 on the underside of the strikethrough plate 601 is aligned with the longitudinal axis of the test sample, and ensure that the fluid port 603 is centered over the pre-marked dose location on the test sample. The central nAMF droplet applied to the test sample should now be visible through the fluid port 603 at the dose location on the test sample.

c) After it is properly positioned over the test sample, gently rest the strikethrough plate 601 over/onto the test sample.

d) Using an adjustable volume pipettor, dispense 2.0 mL of nAMF into the fluid well 608 in the strikethrough plate 601. Dispense the fluid smoothly without splashing, along the conical portion of the wall of the fluid well 608 within a time of 3 seconds or less. When the fluid enters the port 603, electrical connection between the electrodes 604 will be established through the fluid and the circuit interval timer will start timing. When the fluid is acquired by the test sample, electrical connection between the electrodes 604 will be broken and the circuit interval timer will stop timing. Promptly after the circuit interval timer stops timing, start a stopwatch-type timer set to alarm after 2 minutes, leaving the strikethrough plate resting on the test sample during this time. Promptly record the first acquisition time (ACQ-1) displayed on the circuit interval timer, to the nearest 0.1 seconds.

e) After the 2 minutes have elapsed, measure the first Interfacial Free Fluid (IFF-1) as follows.

i) Place the IFF rubber pad onto a horizontal work surface. Weigh a first single fresh sheet of the filter paper for this IFF-1 measurement (IFF-1 filter paper sheet), and record the weight as IFF-1ᵢₙᵢₜᵢₐₗ. Place the IFF-1 filter paper sheet over the IFF rubber pad, squared and centered thereover. Promptly lift and move the strikethrough plate 601 from the test sample to the IFF-1 filter paper sheet such that the plate is squared and centered on the filter paper, and immediately start a stopwatch-type timer set to alarm in 8 minutes.

ii) After 10 seconds have elapsed on the 8 minute timer, remove the strikethrough plate from the IFF-1 filter paper and gently replace it back onto the test sample, exactly as previously positioned.

iii) Within the next 10 seconds, measure the mass of the IFF-1 filter paper to the nearest 0.0001 g and record as IFF-1_{final}.

6) The second acquisition time (ACQ-2) for the test sample is measured as follows.

a) After 8 minutes have elapsed per the previously set timer, using an adjustable volume pipettor, dispense a dose of 4.0 mL of nAMF into the fluid well 608 in the strikethrough plate 601. Dispense the fluid smoothly without splashing, along the conical portion of the wall of the fluid well 608 within a time of 3 seconds or less. Promptly after the circuit interval timer stops timing, start a stopwatch-type timer set to alarm after 2 minutes, leaving the strikethrough plate resting on the test sample during this time. Promptly record the second acquisition time (ACQ-2) displayed on the circuit interval timer, to the nearest 0.1 seconds.

b) After 2 minutes have elapsed, measure the second Interfacial Free Fluid (IFF-2) as follows.

i) Place the IFF rubber pad onto a horizontal work surface. Weigh a second single fresh sheet of the filter paper for this IFF-2 measurement (IFF-2 filter paper sheet) and record the weight as IFF-2ᵢₙₜᵢₐₗ. Place the IFF-2 filter paper sheet over the IFF rubber pad, squared and centered thereover. Promptly lift and move the strikethrough plate 601 from the test sample to the IFF-2 filter paper sheet such that the plate is squared and centered on the filter paper, and immediately start a stopwatch-type timer set to alarm in 8 minutes.

ii) After 10 seconds have elapsed on the 8 minute timer, remove the strikethrough plate from the IFF-2 filter paper and gently replace it back onto the test sample, exactly as previously positioned.

iii) Within the next 10 seconds, measure the mass of the IFF-2 filter paper to the nearest 0.0001 g and record as IFF-2_{final}.

7) The third acquisition time (ACQ-3) is measured as follows.

a) After 8 minutes have elapsed per the previously set timer, using an adjustable volume pipettor dispense a dose of 2.0 mL of nAMF into the fluid well 608 in the strikethrough plate 601. Dispense the fluid smoothly without splashing, along the conical portion of the wall of the fluid well 608 within a time of 3 seconds or less. Promptly after the circuit interval timer stops timing, start a stopwatch-type timer set to alarm after 2 minutes, leaving the strikethrough plate resting on the test sample during this time. Promptly record the third acquisition time (ACQ-3) displayed on the circuit interval timer, to the nearest 0.1 seconds.

b) After 2 minutes have elapsed, measure the second Interfacial Free Fluid (IFF-3) as follows.

i) Place the IFF rubber pad onto a horizontal work surface. Weigh a third single fresh sheet of the filter paper for this IFF-3 measurement (IFF-3 filter paper sheet) and record the weight as IFF-3ᵢₙᵢₜᵢₐₗ. Place the IFF-3 filter paper sheet over the IFF rubber pad, squared and centered thereover. Promptly lift and move the strikethrough plate 601 from the test sample to the IFF-3 filter paper sheet such that the plate is squared and centered on the filter paper, and immediately start a stopwatch-type timer set to alarm in 8 minutes.

ii) After 10 seconds have elapsed on the 8 minute timer, remove the strikethrough plate from the IFF-3 filter paper sheet and set it on its side so that the bottom side of the plate is not contacting the work surface.

iii) Within the next 10 seconds, measure the mass of the IFF-3 filter paper sheet to the nearest 0.0001 g and record as IFF-3_{final}.

8) Measure Surface Free Fluid (SFF) as follows. Weigh a first, neat stack of 5 fresh sheets of the filter paper for this SFF measurement (SFF filter paper stack) and record the weight as SFFᵢₙᵢₜᵢₐₗ. After 8 minutes have elapsed per the previously set timer, place the SFF filter paper stack on top of the wearer-facing side of the test sample such that it is centered over the dose location. Now place the strikethrough plate 601 on top of the SFF filter paper stack such that the bottom side of the plate is centered on the filter paper stack, and immediately start a stopwatch-type timer set to alarm in 10 seconds. After 10 seconds have elapsed, remove the strikethrough plate 601 from the filter paper stack and set it aside. Measure the mass of the SFF filter paper stack to the nearest 0.0001 g and record as SFF_{final}. Immediately proceed to the next step.

9) Measure overall rewet as follows. Weigh a second, neat stack of 5 fresh sheets of the filter paper for this REWET measurement (REWET filter paper stack) and record weight as REWETᵢₙᵢₜᵢₐₗ. Place the REWET filter paper stack on top of the wearer-facing side of the test sample such that it is centered over the dose location. Now place the rewet weight assembly on top of the REWET filter paper stack such that the weight is centered on the stack, and immediately start a stopwatch-type timer set to alarm in 30 seconds. After 30 seconds have elapsed, remove the rewet weight assembly and measure the mass of REWET filter paper stack to the nearest 0.0001 g, then record as REWET_{final}.

10) Discard the test sample and thoroughly clean and then dry the strikethrough plate 601 including the fluid well 608, fluid port 603, longitudinal fluid channel 607 and the bottom surface, prior to testing the next sample.

11) Make the following calculations for each of the parameters measured, as follows. Calculate IFF-1 by subtracting IFF-1ᵢₙᵢₜᵢₐₗ from IFF-1_{final}, and record to the nearest 0.0001 g. Calculate IFF-2 by subtracting IFF-2ᵢₙᵢₜᵢₐₗ from IFF-2_{final}, and record to the nearest 0.0001 g. Calculate IFF-3 by subtracting IFF-3ᵢₙᵢₜᵢₐₗ from IFF-3_{final}, and record to the nearest 0.0001 g. Calculate SFF by subtracting SFFᵢₙᵢₜᵢₐₗ from SFF_{final}, and record to the nearest 0.0001 g. Calculate Overall Rewet by subtracting REWETᵢₙᵢₜᵢₐₗ from REWET_{final}, and record to the nearest 0.0001 g.

The entire procedure is repeated for a total of three replicate test samples. The reported value for each of the parameters is the average of the three individually recorded measurements for each Acquisition Time (ACQ-1, ACQ-2 and ACQ-3) to the nearest 0.1 seconds, Interfacial Free Fluid (IFF-1, IFF-2 and IFF-3) to the nearest 0.0001 g, Surface Free Fluid (SFF) to the nearest 0.0001 g and Overall Rewet to the nearest 0.0001 g.

### New Artificial Menstrual Fluid (nAMF) Preparation

New Artificial Menstrual Fluid (nAMF) is a mixture of defibrinated sheep blood, a phosphate buffered saline solution and a mucous component. The nAMF is prepared such that it has a viscosity between 7.40 to 9.00 centipoise at 23° C.

Viscosity of the nAMF is measured using a low viscosity rotary viscometer (a suitable instrument is the Brookfield DV2T fitted with a Brookfield UL adapter, available from AMETEK Brookfield, Middleboro, Mass., or equivalent). The appropriate size spindle for the viscosity range is selected, and the instrument is operated and calibrated as per the manufacturer. Measurements are taken at 23° C.±1 C.° and at 60 rpm. Results are reported to the nearest 0.01 centipoise.

Reagents needed for the nAMF preparation include: defibrinated sheep blood with a packed cell volume of 38% or greater (collected under sterile conditions, available from Cleveland Scientific, Inc., Bath, Ohio, or suitably comparable source), gastric mucin with a viscosity target of 3-4 centistokes when prepared as a 2% aqueous solution (crude form, sterilized, available from American Laboratories, Inc., Omaha, Nebr., or suitably comparable source), sodium phosphate dibasic anhydrous (reagent grade), sodium chloride (reagent grade), sodium phosphate monobasic monohydrate (reagent grade), sodium benzoate (reagent grade), benzyl alcohol (reagent grade) and distilled water, each available from VWR International or suitably comparable source.

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add distilled water to volume. Mix thoroughly. To prepare about 200 mL of phosphate buffered saline solution, add 49.50 g±0.10 g of Solution A and 157.50 g±0.10 g of Solution B to a sufficiently size bottle that has a lid with a good seal. Then add 1.0 g of sodium benzoate and 1.60 g of benzyl alcohol to the bottle along with a stir bar and set aside.

The mucous component of the nAMF is a mixture of the phosphate buffered saline solution and gastric mucin. The amount of gastric mucin added to the mucous component directly affects the final viscosity of the prepared nAMF. To determine the amount of gastric mucin needed to achieve nAMF within the target viscosity range (7.4-9.0 centipoise at 23° C. and 60 rpm), prepare 3 batches of nAMF with varying amounts of gastric mucin in the mucous component, and then interpolate the exact amount needed from a concentration versus viscosity curve with a least squares linear fit through the three points. A successful range of gastric mucin is usually between 13 to 15 grams per 400 mL batch of nAMF, although this can vary significantly based upon the supplier, age, and lot (production batch) of mucin.

To prepare about 200 mL of the mucous component, add the pre-determined amount of gastric mucin to the bottle containing the previously prepared phosphate buffered solution and then apply the lid. Place the bottle on a wrist-action shaker for 5 minutes at the highest speed. After 5 minutes, remove the flask of mucous component from the wrist-action shaker and place onto a magnetic stir plate. Stir for at least 2 hours until there are no lumps of mucin present, then remove the stir bar from the flask. Using a homogenizer, blend the mucous component for 5 minutes at 10,000 rpm. A suitable homogenizer is the T18 Ultra-Turrax fitted with a S18N-19G dispersing tool (19 mm stator diameter, 12.7 mm rotor diameter, 0.4 mm gap between rotor and stator), both available from IKA Works, Inc, Wilmington, N.C., or suitably comparable source. After the final mixing step, measure and record the viscosity of the mucous component to the nearest 0.01 centipoise at 23° C.±1 C.° and at 20 rpm using the viscometer with the UL adapter. Ensure that the viscosity of the prepared mucous component is within the target range of 9.0-11.0 centipoise.

The nAMF is a 50:50 mixture of the mucous component and sheep blood. Ensure the temperature of the sheep blood and mucous component are 23° C.±1 C° . To prepare about 400 mL of nAMF, add 200 g of the mucous component to a glass bottle with at least 500 mL capacity. Now add 200 g of sheep blood to the bottle along with a stir bar. Mix on a magnetic stir plate until thoroughly combined. Ensure the viscosity of the prepared nAMF is within the target range of 7.4-9.0 centipoise when measured at 23° C.±1 C.° and 60 rpm using the viscometer with the UL adapter. If the viscosity is too high, it can be adjusted by adding the previously prepared phosphate buffered saline solution in 0.5 g increments followed by stirring for 2 minutes and then re-checking the viscosity until the target range is reached.

The qualified nAMF should be refrigerated at 4° C. unless intended for immediate use. nAMF may be stored in an air-tight container at 4° C. for up to 48 hours after preparation. Prior to testing, the nAMF must be brought to 23° C.±1 C° . Any unused portion is discarded after testing is complete.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “approximately 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

In view of the description above, the following non-limiting examples are contemplated. Any of these examples as well as others may be claimed in one or more subsequent non-provisional patent applications based in whole or in part on the disclosure herein:
1. A feminine hygiene pad (10), comprising a liquid permeable topsheet (20) comprising a fibrous nonwoven web, a fibrous fluid management layer (30) beneath the topsheet, an absorbent structure (40) beneath the fluid management layer, and a backsheet (50) beneath the absorbent structure, wherein:
   the fibrous nonwoven web comprises bicomponent staple topsheet fibers, wherein the topsheet fibers:
      have an average denier of 1.5 to 2.5;
      have a sheath-core configuration, wherein the sheath component comprises polyethylene (PE) and the core component comprises polyethylene terephthalate (PET), in a weight ratio of PE:PET of 40:60 to 60:40; and
      comprise a blend of hydrophilic fibers and hydrophobic fibers, in a weight ratio of hydrophilic fibers to hydrophobic fibers of about 30:70 to 70:30, more preferably about 35:65 to 65:35, and even more preferably about 40:60 to 60:40, wherein the hydrophilicity of the hydrophilic fibers is effected by application of a surface treatment composition;
   the fibrous nonwoven web has a basis weight of about 18 gsm to 40 gsm, more preferably about 20 gsm to 30 gsm, even more preferably about 22 gsm to 26 gsm;
      the fibrous nonwoven web comprises a plurality of randomly-distributed inter-fiber bonds wherein sheaths of adjacent fibers are fusion-bonded together without compression; and
      wherein the topsheet bears a topical application of an anti-stick agent.
2. The feminine hygiene pad of example 1 wherein the fluid management layer (30) comprises carded staple fibers including absorbent fibers of regenerated cellulose in a weight fraction of the fluid management layer of about 10 percent to about 60 percent, bicomponent stiffening fibers in a weight fraction of the fluid management layer of about 25 percent to about 70 percent, and resilient fibers in a weight fraction of the fluid management layer of about 15 percent to about 70 percent.
3. The feminine hygiene pad of example 2 wherein the absorbent fibers are about 1 dtex to 7 dtex, or more preferably about 1.4 dtex to 6 dtex, or even more preferably about 1.7 dtex to 5 dtex.
4. The feminine hygiene pad of example 2 wherein the absorbent fibers are about 0.6 to 2.4 dtex, more preferably about 0.9 to 2.1 dtex, even more preferably about 1.1 to 1.9 dtex, and most preferably about 1.3 to 1.7 dtex.
5. The feminine hygiene pad of any of examples 2-4 wherein the stiffening fibers are about 1.0 dtex to 6 dtex, more preferably about 1.5 dtex to 5 dtex, or even more preferably about 2.0 dtex to 4 dtex.
6. The feminine hygiene pad of any of examples 2-5 wherein the stiffening fibers have a sheath-core configuration.
7. The feminine hygiene pad of example 6 wherein the core component comprises PET.
8. The feminine hygiene pad of either of examples 6 or 7 wherein the sheath component comprises PE.
9. The feminine hygiene pad of any of examples 2-8 wherein the resilient fibers are about 4 dtex to 15 dtex, or more preferably about 5 dtex to 12 dtex, or even more preferably about 6 dtex to 10 dtex.
10. The feminine hygiene pad of example 9 wherein the resilient fibers comprise a polymer selected from the group consisting of polypropylene (PP), polyethylene (PE), polyethylene terephthalate (PET), and combinations thereof.
11. The feminine hygiene pad of any of examples 2-10 wherein the resilient fibers are bicomponent fibers.
12. The feminine hygiene pad of example 11 wherein the resilient fibers have a sheath-core configuration.
13. The feminine hygiene pad of example 12 wherein the core component comprises PP and/or PET and the sheath component comprises PP and/or PE.
14. The feminine hygiene pad of any of the preceding examples wherein the fluid management layer comprises a plurality of randomly-distributed inter-fiber bonds wherein adjacent fibers are fusion-bonded together without compression.
15. The feminine hygiene pad of any of the preceding examples wherein the fluid management layer comprises a plurality of strata.
16. The feminine hygiene pad of any of the preceding examples wherein fibers of the fluid management layer are integrated in a z-direction.
17. The feminine hygiene pad of any of the previous examples wherein the topsheet has a pattern of apertures therethrough.
18. The feminine hygiene pad of example 17 wherein the apertures have an average area of 0.5 mm² to 2.5 mm², preferably 0.6 mm² to 1.2 mm².
19. The feminine hygiene pad of either of examples 17 or 18 wherein the apertures collectively constitute an open area 6 percent to 25 percent, more preferably 8 percent to 18 percent, and even more preferably 10 percent to 15 percent.
20. The feminine hygiene pad of any of the preceding examples wherein the anti-stick agent comprises a polypropylene glycol material, and optionally, a carrier.
21. The feminine hygiene pad of example 20 wherein the polypropylene glycol material is selected from the group consisting of polypropylene glycol copolymer, polypropylene glycol surfactant, and mixtures thereof.
22. The feminine hygiene pad of example 21 wherein the polypropylene glycol material is polypropylene glycol copolymer, and wherein said polypropylene glycol copolymer comprises an internal block component and a terminal block component, wherein said internal block component has a formula:

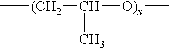

and said terminal block component has a formula:

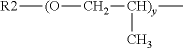

wherein x is from 2 to 120, y is from 2 to 100, and R2 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate.
23. The feminine hygiene pad of example 21 wherein the polypropylene glycol material is polypropylene glycol copolymer, and wherein said polypropylene glycol copolymer is selected from the group consisting of PPG-12 dimethicone, bis-PPG-15 dimethicone/IPDI copolymer, PPG/polycaprolactone block copolymer, PPG/polybutanediol/PEG triblock copolymer, polyethylimine/PPG copolymer; polyacrylic acid-g-PPG graft copolymer, and mixtures thereof.
24. The feminine hygiene pad of example 21 wherein the polypropylene glycol material is polypropylene glycol surfactant, and wherein said polypropylene glycol surfactant has a formula:

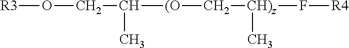

wherein R3 is hydrogen, alkyl, alkyl carbonyl, alkylenelamine, alkylenelamide, alkylene phosphate, alkylene carboxylic acid, alkylene sulfonate salt or alkylene quat with a maximum number of carbon elements of less than or equal to 6; R4 is octyl, nonyl, decyl, iosdecyl, lauryl, myristyl, cetyl, isohexadecyl, oleyl, stearyl, isostearyl, tallowoyl, linoleyl, jojoba, lanolin, behenyl, C24-C28 alkyl, C30-C45 alkyl, dinonylphenyl, dodecyl phenyl, or soya; z is from 1 to 100; and F is a functional group selected from the group consisting of an ether group, an ester group, an amine group, an amide group, and a phosphate ester group.
25. The feminine hygiene pad of example 21 wherein the polypropylene glycol material is polypropylene glycol surfactant, and wherein said polypropylene glycol surfactant has a formula:

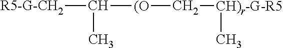

wherein R5 is hexyl, 2-ethylhexyl, octyl, nonyl, decyl, isodecyl, lauryl, cocoyl, myristyl, cetyl, isohexadecyl, oleyl, stearyl, isostearyl, tallow, linoleyl, octyl phenyl, or nonyl phenyl; r is from 1 to 120; and G is an ether, an ester, an amine, or an amide linkage.
26. The feminine hygiene pad of example 21 wherein the polypropylene glycol material is polypropylene glycol surfactant, and wherein said polypropylene glycol surfactant is selected from the group consisting of PPG-30 cetyl ether, PPG-20 methyl glucose ether distearate, PPG-20 methyl glucose ether acetate, PPG-20 sorbitan tristearate, PPG-20 methyl glucose ether distearate, PPG-20 distearate, PPG-15 stearyl ether, PPG-15 stearyl ether benzoate, PPG-15 isohexadecyl ether, PPG-15 stearate, PPG-15 dicocoate, PPG-12 dilaurate, PPG-11 stearyl ether, PPG-10 cetyl ether, PPG-10 glyceryl stearate, PPG-10 sorbitan monosterate, PPG-10 hydrogenated castor oil, PPG-10 cetyl phosphate, PPG-10 tallow amine, PPG-10 oleamide, PPG-10 cetyl ether phosphate, PPG-10 dinonylphenolate, PPG-9 laurate, PPG-8 dioctate, PPG-8 diethylhexylate, PPG-7 lauryl ether, PPG-5 lanolin wax ether, PPG-5 sucrose cocoate, PPG-5 lanolin wax, PPG-4 jojoba alcohol ether, PPG-4 lauryl ether, PPG-3 myristyl ether, PPG-3 myristyl ether propionate, PPG-3 benzyl ether myristate, PPG-3 hydrogenated castor oil, PPG-3-hydroxyethyl soyamide, PPG-2 cocamide, PPG-2 lanolin alcohol ether, PPG-1 coconut fatty acid isopropanolamide, and mixtures thereof.
27. The feminine hygiene pad of example 26 wherein the polypropylene glycol surfactant is PPG-15 stearyl ether.
28. The feminine hygiene pad of claim 20 wherein the polypropylene glycol material is polypropylene glycol homopolymer, and wherein said polypropylene glycol homopolymer has a formula:

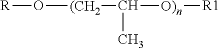

wherein R is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; R1 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and n is from 3 to 160.
29. The feminine hygiene pad of claim 20 wherein the polypropylene glycol material is polypropylene glycol.
30. The feminine hygiene pad of example 29 wherein the polypropylene glycol has a number average molecular weight of from about 400 to about 10,000.
31. The feminine hygiene pad of any of examples 20-30 wherein the carrier comprises caprylic/capric triglyceride.
32. The absorbent article of any of the preceding examples, wherein the anti-stick agent consists essentially of a polypropylene glycol material and a carrier.
33. A feminine hygiene pad (10), comprising a liquid permeable topsheet (20) comprising a fibrous nonwoven web, a fibrous fluid management layer (30) beneath the topsheet, an absorbent structure (40) beneath the fluid management layer, and a backsheet (50) beneath the absorbent structure, wherein:
   the fibrous nonwoven web comprises bicomponent staple topsheet fibers, wherein the topsheet fibers:
      have an average denier of 1.5 to 2.5;
      have a sheath-core configuration, wherein the sheath component comprises polyethylene (PE) and the core component comprises polyethylene terephthalate (PET), in a weight ratio of PE:PET of 40:60 to 60:40; and
      in predominant weight fraction of the fibrous nonwoven web, and preferably, all, are hydrophobic;
   the fibrous nonwoven web has a basis weight of about 18 gsm to 40 gsm, more preferably about 20 gsm to 30 gsm, even more preferably about 22 gsm to 26 gsm;
      the fibrous nonwoven web comprises a plurality of randomly-distributed inter-fiber bonds wherein sheaths of adjacent fibers are fusion-bonded together without compression; and
      the topsheet has a pattern of apertures therethrough.
34. The feminine hygiene pad of example 33 wherein the apertures have an average size of 0.5 mm² to 2.5 mm², preferably 0.6 mm² to 1.2 mm².
35. The feminine hygiene pad of either of examples 33 or 34 wherein the apertures constitute an open area of 6 percent to 25 percent, more preferably 8 percent to 18 percent, and even more preferably 10 percent to 15 percent.
36. The feminine hygiene pad of any of examples 33-35 wherein the topsheet bears a topical application of an anti-stick agent.
37. The feminine hygiene pad of example 36 wherein the anti-stick agent comprises a polypropylene glycol material, and optionally, a carrier.
38. The feminine hygiene pad of example 37 wherein the polypropylene glycol material is selected from the group consisting of polypropylene glycol copolymer, polypropylene glycol surfactant, and mixtures thereof.
39. The feminine hygiene pad of example 38 wherein the polypropylene glycol material is polypropylene glycol copolymer, and wherein said polypropylene glycol copolymer comprises an internal block component and a terminal block component, wherein said internal block component has a formula:

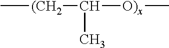

and said terminal block component has a formula:

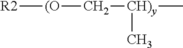

wherein x is from 2 to 120, y is from 2 to 100, and R2 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate.
40. The feminine hygiene pad of example 38 wherein the polypropylene glycol material is polypropylene glycol copolymer, and wherein said polypropylene glycol copolymer is selected from the group consisting of PPG-12 dimethicone, bis-PPG-15 dimethicone/IPDI copolymer, PPG/polycaprolactone block copolymer, PPG/polybutanediol/PEG triblock copolymer, polyethylimine/PPG copolymer; polyacrylic acid-g-PPG graft copolymer, and mixtures thereof.
41. The feminine hygiene pad of example 38 wherein the polypropylene glycol material is polypropylene glycol surfactant, and wherein said polypropylene glycol surfactant has a formula:

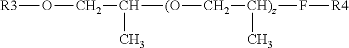

wherein R3 is hydrogen, alkyl, alkyl carbonyl, alkylenelamine, alkylenelamide, alkylene phosphate, alkylene carboxylic acid, alkylene sulfonate salt or alkylene quat with a maximum number of carbon elements of less than or equal to 6; R4 is octyl, nonyl, decyl, iosdecyl, lauryl, myristyl, cetyl, isohexadecyl, oleyl, stearyl, isostearyl, tallowoyl, linoleyl, jojoba, lanolin, behenyl, C24-C28 alkyl, C30-C45 alkyl, dinonylphenyl, dodecyl phenyl, or soya; z is from 1 to 100; and F is a functional group selected from the group consisting of an ether group, an ester group, an amine group, an amide group, and a phosphate ester group.
42. The feminine hygiene pad of example 38 wherein the polypropylene glycol material is polypropylene glycol surfactant, and wherein said polypropylene glycol surfactant has a formula:

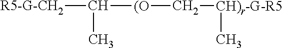

wherein R5 is hexyl, 2-ethylhexyl, octyl, nonyl, decyl, isodecyl, lauryl, cocoyl, myristyl, cetyl, isohexadecyl, oleyl, stearyl, isostearyl, tallow, linoleyl, octyl phenyl, or nonyl phenyl; r is from 1 to 120; and G is an ether, an ester, an amine, or an amide linkage.
43. The feminine hygiene pad of example 38 wherein the polypropylene glycol material is polypropylene glycol surfactant, and wherein said polypropylene glycol surfactant is selected from the group consisting of PPG-30 cetyl ether, PPG-20 methyl glucose ether distearate, PPG-20 methyl glucose ether acetate, PPG-20 sorbitan tristearate, PPG-20 methyl glucose ether distearate, PPG-20 distearate, PPG-15 stearyl ether, PPG-15 stearyl ether benzoate, PPG-15 isohexadecyl ether, PPG-15 stearate, PPG-15 dicocoate, PPG-12 dilaurate, PPG-11 stearyl ether, PPG-10 cetyl ether, PPG-10 glyceryl stearate, PPG-10 sorbitan monosterate, PPG-10 hydrogenated castor oil, PPG-10 cetyl phosphate, PPG-10 tallow amine, PPG-10 oleamide, PPG-10 cetyl ether phosphate, PPG-10 dinonylphenolate, PPG-9 laurate, PPG-8 dioctate, PPG-8 diethylhexylate, PPG-7 lauryl ether, PPG-5 lanolin wax ether, PPG-5 sucrose cocoate, PPG-5 lanolin wax, PPG-4 jojoba alcohol ether, PPG-4 lauryl ether, PPG-3 myristyl ether, PPG-3 myristyl ether propionate, PPG-3 benzyl ether myristate, PPG-3 hydrogenated castor oil, PPG-3-hydroxyethyl soyamide, PPG-2 cocamide, PPG-2 lanolin alcohol ether, PPG-1 coconut fatty acid isopropanolamide, and mixtures thereof.
44. The feminine hygiene pad of example 43 wherein the polypropylene glycol surfactant is PPG-15 stearyl ether.
45. The feminine hygiene pad of claim 37 wherein the polypropylene glycol material is polypropylene glycol homopolymer, and wherein said polypropylene glycol homopolymer has a formula:

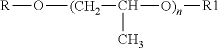

wherein R is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; R1 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and n is from 3 to 160.
46. The feminine hygiene pad of claim 37 wherein the polypropylene glycol material is polypropylene glycol.
47. The feminine hygiene pad of example 46 wherein the polypropylene glycol has a number average molecular weight of from about 400 to about 10,000.
48. The feminine hygiene pad of any of examples 37-47 wherein the carrier comprises caprylic/capric triglyceride.
49. The absorbent article of any of examples 36-48, wherein the anti-stick agent consists essentially of a polypropylene glycol material and a carrier.

What is claimed is:

1. A feminine hygiene pad, comprising a liquid permeable topsheet comprising a fibrous nonwoven web, a fibrous fluid management layer beneath the topsheet, an absorbent structure beneath the fluid management layer, and a backsheet beneath the absorbent structure, wherein:
the fibrous nonwoven web comprises bicomponent staple topsheet fibers, wherein the topsheet fibers:
have an average denier of 1.5 to 2.5;
have a sheath-core configuration, wherein the sheath component comprises polyethylene (PE) and the core component comprises polyethylene terephthalate (PET), in a weight ratio of PE:PET of 40:60 to 60:40; and
comprise a blend of hydrophilic fibers and hydrophobic fibers, in a weight ratio of hydrophilic fibers to hydrophobic fibers of about 30:70 to 70:30, wherein the hydrophilicity of the hydrophilic fibers is effected by application of a surface treatment composition;
the fibrous nonwoven web has a basis weight of about 18 gsm to 40 gsm;
the fibrous nonwoven web comprises a plurality of randomly-distributed inter-fiber bonds wherein sheaths of adjacent fibers are fusion-bonded together without compression; and
wherein the topsheet bears a topical application of an anti-stick agent.

2. The feminine hygiene pad of claim 1 wherein the fluid management layer comprises carded staple fibers including absorbent fibers of regenerated cellulose in a weight fraction of the fluid management layer of about 10 percent to about 60 percent, bicomponent stiffening fibers in a weight fraction of the fluid management layer of about 25 percent to about 70 percent, and resilient fibers in a weight fraction of the fluid management layer of about 15 percent to about 70 percent.

3. The feminine hygiene pad of claim 2 wherein the absorbent fibers are about 0.6 to 2.4 dtex.

4. The feminine hygiene pad of claim 2 wherein the stiffening fibers are about 1.0 dtex to 6 dtex.

5. The feminine hygiene pad of claim 2 wherein the stiffening fibers are bicomponent fibers having a sheath-core configuration, wherein a core component comprises PET.

6. The feminine hygiene pad of claim 2 wherein the resilient fibers are about 4 dtex to 15 dtex.

7. The feminine hygiene pad of claim 6 wherein the resilient fibers comprise a polymer selected from the group consisting of polypropylene (PP), polyethylene (PE), polyethylene terephthalate (PET), and combinations thereof.

8. The feminine hygiene pad of claim 2 wherein the resilient fibers are bicomponent fibers having a sheath-core configuration.

9. The feminine hygiene pad of claim 1 wherein the fluid management layer comprises a plurality of randomly-distributed inter-fiber bonds wherein adjacent fibers are fusion-bonded together without compression.

10. The feminine hygiene pad of claim 1 wherein the fluid management layer comprises a plurality of strata.

11. The feminine hygiene pad of claim 10 wherein fibers of the fluid management layer are integrated in a z-direction.

12. The feminine hygiene pad of claim 1 wherein the topsheet has a pattern of apertures therethrough.

13. The feminine hygiene pad of claim 12 wherein the apertures have an average area of 0.5 mm2 to 2.5 mm2.

14. The feminine hygiene pad of claim 12 wherein the apertures collectively constitute an open area 6 percent to 25 percent.

15. The feminine hygiene pad of claim 1 wherein the anti-stick agent comprises a polypropylene glycol material.

16. The feminine hygiene pad of claim 15 wherein the polypropylene glycol material is polypropylene glycol.

17. A feminine hygiene pad, comprising a liquid permeable topsheet comprising a fibrous nonwoven web, a fibrous fluid management layer beneath the topsheet, an absorbent structure beneath the fluid management layer, and a backsheet beneath the absorbent structure, wherein:
the fibrous nonwoven web comprises bicomponent staple topsheet fibers, wherein the topsheet fibers:
have an average denier of 1.5 to 2.5;
have a sheath-core configuration, wherein the sheath component comprises polyethylene (PE) and the core component comprises polyethylene terephthalate (PET), in a weight ratio of PE:PET of 40:60 to 60:40; and
in predominant weight fraction of the fibrous nonwoven web, are hydrophobic;
the fibrous nonwoven web has a basis weight of about 18 gsm to 40 gsm;
the fibrous nonwoven web comprises a plurality of randomly-distributed inter-fiber bonds wherein sheaths of adjacent fibers are fusion-bonded together without compression; and
the topsheet has a pattern of apertures therethrough.

18. The feminine hygiene pad of claim 17 wherein the apertures have an average size of 0.5 mm2 to 2.5 mm2.

19. The feminine hygiene pad of claim 17 wherein the apertures constitute an open area of 6 percent to 25 percent.

20. The feminine hygiene pad of claim 17 wherein the topsheet bears a topical application of an anti-stick agent, wherein the anti-stick agent comprises a polypropylene glycol material.
